(19) United States
(12) Patent Application Publication
(10) Pub. No.: US 2003/0190653 A1
(43) Pub. Date: Oct. 9, 2003

(54) Regulated gene in the pathophysiology of ischemic stroke

(76) Inventors: Mehrdad Shamloo, Foster City, CA (US); Mirella Gonzalez-Zulueta, Pacifica, CA (US); Tadeusz Wieloch, Lund (SE)

Correspondence Address: BOZICEVIC, FIELD & FRANCIS LLP, 200 MIDDLEFIELD RD, SUITE 200, MENLO PARK, CA, 94025, US

(21) Appl. No.: 10/340,123

(22) Filed: Jan. 10, 2003

Related U.S. Application Data
(60) Provisional application No. 60/347,437, filed on Jan. 11, 2002.
(60) Provisional application No. 60/381,628, filed on May 17, 2002.

(51) Int. Cl. C12Q 1/68; A01K 67/00; C07H 21/04; C12N 9/00; C12P 21/02; C12N 5/06

(52) U.S. Cl. 435/6; 435/69.1; 435/320.1; 435/368; 435/183; 530/350; 536/23.2; 800/8

(57) ABSTRACT

The present invention identifies the K11 gene, whose gene products can be modulated to provide a protective effect against stroke, especially ischemic stroke, epilepsy and neurodegenerative disorders and enhancement of memory function. Further, the invention provides methods for diagnosing or assessing an individual's susceptibility to a stroke. Also provided are therapeutic methods for treating a stroke patient or methods for prophylactically treating an individual susceptible to stroke. Additionally, the invention describes screening methods for identifying agents that can be administered to treat individuals that have suffered a stroke or that are at risk for stroke.

### INTRODUCTION

Neurodegenerative diseases are characterized by the dysfunction and death of neurons, leading to the loss of neurologic functions mediated by the brain, spinal cord and the peripheral nervous system. These disorders have a major impact on society. For example, approximately 4 to 5 million Americans are afflicted with the chronic neurodegenerative disease known as Alzheimer's disease. Other examples of chronic neurodegenerative diseases include diabetic peripheral neuropathy, multiple sclerosis, amyotrophic lateral sclerosis, Huntington's disease and Parkinson's disease. Normal brain aging is also associated with loss of normal neuronal function and may entail the depletion of certain neurons.

Though the mechanisms responsible for the dysfunction and death of neurons in neurodegenerative disorders are not well understood, a common theme is that loss of neurons results in both the loss of normal functions and the onset of adverse behavioral symptoms. Therapeutic agents that have been developed to retard loss of neuronal activity and survival has been largely ineffective. Some have toxic side effects that limit their usefulness. Other promising therapies, such as neurotrophic factors, are prevented from reaching their target site because of their inability to cross the blood-brain barrier.

Stroke is the third ranking cause of death in the United States, and accounts for half of neurology inpatients. Depending on the area of the brain that is damaged, a stroke can cause coma, paralysis, speech problems and dementia. The five major causes of cerebral infarction are vascular thrombosis, cerebral embolism, hypotension, hypertensive hemorrhage, and anoxia/hypoxia.

The brain requires glucose and oxygen to maintain neuronal metabolism and function. Hypoxia refers to inadequate delivery of oxygen to the brain, and ischemia results from insufficient cerebral blood flow. The consequences of cerebral ischemia depend on the degree and duration of reduced cerebral blood flow.

Mechanisms of hypoxic sensing and response may have been established early in evolutionary history. The majority of the transcription responses to hypoxia are mediated by hypoxia-inducible factor (HIF) complex. HIF-1 is a heterodimeric bHLH-PAS transcription factor that mediates changes in gene expression in response to changes in oxygen concentration. HIF-1 is required for cardiac and vascular development and may mediate adaptation to hypoxia that is critical for tumor progression (see Semenza (1999) Annu. Rev. Cell.) When cellular oxygen levels are high, HIF-1 is targeted for ubiquitination and proteosomal degradation. However, in hypoxic conditions, degradation of HIF is inhibited. This permits HIF to translocate to the nucleus, dimerize with ARNT, and activate the expression of target genes, which act to increase oxygen delivery or implement metabolic adaptation to hypoxia. Despite intensive study, the molecular mechanisms by which cellular oxygen levels are sensed are not well understood.

One mechanism that may be involved in oxygen sensing utilizes the PAS domain, which is present in HIF-1, as well as aryl hydrocarbon receptor (AHR), and single minded (SIM) protein. The PAS domain is a signaling module found in proteins from bacteria to humans. PAS domains are found in a wide range of proteins including histidine and serine/threonine kinases, chemoreceptors and photoreceptors for taxis and tropism, circadian clock proteins, voltage-activated potassium channels, cyclic nucleotide phosphodiesterases, and regulators of responses to hypoxia and embryological development of the central nervous system. Members of one group of PAS domain proteins also contain basic helix-loop-helix (bHLH) motifs in addition to the PAS domains and act as transcription factors. (See Taylor, B. L. and Zhulin, I. B. (1999) Microbiol. Mol. Biol. Rev. 63: 479-506; and Crews, S. T. and Fan, C- M. (1999) Curr. Opin. Gen. Dev. 9: 580-587 for review).

Neurons can tolerate ischemia for 30-60 minutes, but perfusion must be reestablished before 3-6 hours of ischemia have elapsed. Neuronal damage can be less severe and reversible if flow is restored within a few hours, providing a window of opportunity for intervention. If flow is not reestablished to the ischemic area, a series of metabolic processes ensue. The neurons become depleted of ATP and switch over to anaerobic glycolysis (Yamane et al. (2000) J Neurosci Methods 103(2):163-71). Lactate accumulates and the intracellular pH decreases. Without an adequate supply of ATP, membrane ion pumps fail. There is an influx of sodium, water, and calcium into the cell. The excess calcium is detrimental to cell function and contributes to membrane lysis. Cessation of mitochondrial function signals neuronal death (Reichert et al. (2001) J Neurosci. 21(17):6608-16). The astrocytes and oligodendroglia are slightly more resistant to ischemia, but their demise follows shortly if blood flow is not restored (Sochocka et al. (1994) Brain Res 638(1-2):21-8)

Evidence is also emerging in support of the possibility that acute inflammatory reactions to brain ischemia are causally related to brain damage. The inflammatory condition consists of cells (neutrophils at the onset and later monocytes) and mediators (cytokines, chemokines, others). Upregulation of proinflammatory cytokines, chemokines and endothelial-leukocyte adhesion molecules in the brain follow soon after an ischemic insult and at a time-when the cellular component is evolving. The significance of the inflammatory response to brain ischemia is not fully understood (Feuerstein et al. (1997) Ann NY Acad Sci 825:179-93).

In animal models of middle cerebral artery occlusion (MCAO), it has been found that an ischemic penumbra surrounds a focus of dense cerebral ischemia. The ischemic penumbra is the region where cerebral blood flow reduction has exceeded the threshold for failure of electrical function but not that for membrane failure. The ischemic core region enlarges when adjacent, formerly penumbral, areas undergo irreversible deterioration during the initial hours of vascular occlusion. At the same time, the residual penumbra becomes restricted to the periphery of the ischemic territory, and its fate may depend critically upon early therapeutic intervention.

Electrophysiological measurements show penumbral cell depolarizations, associated with an increased metabolic workload, which induce episodes of tissue hypoxia. The frequency of their occurrence correlates with the final volume of ischemic injury. Therefore, penumbral depolarizations have been thought to be important in the pathogenesis of ischemic brain injury. Periinfarct direct current deflections can be suppressed by NMDA Receptor and non-NMDA Receptor antagonists, resulting in a significant reduction of infarct size (Back (1998) Cell Mol Neurobiol. 18(6):621-38). The histopathological sequel within the penumbra consists of various degrees of scattered neuronal injury, also termed “incomplete infarction.” (Lassen (1984) Stroke 15(4):755-8) The reduction of neuronal density at the infarct border is a flow- and time-dependent event, which is affected by the activity of astrocytes and glial cells. Thus, the penumbra is a spatially dynamic brain region of limited viability, which is characterized by complex pathophysiological changes in response to local ischemic injury.

The treatment of stroke includes preventive therapies, such as antihypertensive and antiplatelet drugs, which control and reduce blood pressure and thus reduce the likelihood of stroke. Also, the development of thrombolytic drugs such as t-PA (tissue plasminogen activator) has provided a significant advance in the treatment of ischemic stroke victims, although to be effective it is necessary to begin treatment very early, within about three hours after the onset of symptoms. These drugs dissolve blood vessel clots which block blood flow to the brain and which are the cause of approximately 80% of strokes (see for reviews, Kent et al. (2001) Stroke 32(10):2318-27; and Albers (2001) Neurology 57(5 Suppl 2):S77-81). However, these drugs can also present the side effect of increased risk of bleeding, and t-PA has recently been shown to have direct neurotoxic effects (Flavin et al. (2000) Glia 29(4):347-54). Various neuroprotectors, such as calcium channel antagonists, can sometimes stop damage to the brain as a result of ischemic insult (Horn et al. (2001) Stroke 32(2):570-6). The window of treatment for these drugs is typically broader than that for the clot dissolvers and they do not increase the risk of bleeding.

Studies in in vivo animal models of stroke, as well as in in vitro paradigms of ischemia-induced neuronal death, have shown that damage and dysfunction of neurons following ischemia is dependent on protein-synthesis (Jin et al. (2001) Ann Neurol. 50(1):93-103; Koistinaho et al. (1997) Neuroreport 8(2);i-viii). Thus, general protein-synthesis inhibitors such as cycloheximide, and gene transcription blockers prevent ischemia-induced neuronal death (Snider et al. (2001) Brain Res 917(2):147-57). Therefore, the pathophysiology of ischemic stroke involves regulation of gene expression that ultimately results in neuronal death.

In view of the importance of cerebral ischemia for human mortality and morbidity, the identification of genes involved in the disease, and development of methods of treatment is of great interest.

Literature

A PAS domain protein is described in International Patent Application WO0210200.

### SUMMARY OF THE INVENTION

The present invention relates to novel genetic sequences and methods of use thereof for the diagnosis and treatment of neurologic disorders, including but not limited to focal or global ischemia of the brain and central nervous system. Specifically, a novel gene is identified and described that is differentially expressed in ischemic neuronal tissue, relative to its expression in normal, or non-disease states. The invention also provides methods for the identification of compounds that modulate the expression of the gene or the activity of the gene products, as well as methods for the treatment of disease by administering such compounds to individuals exhibiting neuronal ischemia symptoms. The nucleic acid compositions find use in identifying homologous or related genes; in producing compositions that modulate the effects of ischemia; for gene therapy; mapping functional regions of the encoded protein; and in studying associated physiological pathways.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts in situ hybridization with K11 after 2 hours of MCAO. Rat brain coronal sections were hybridized with K11 RNA probes, after two hours of MCAO and 0-48 hours of recovery. An upregulation of K11 was observed during recovery in regions of the brain sensitive to ischemia.

FIG. 2 depicts in situ hybridization with K11 after 3 minutes of ischemia. Rat brain coronal sections were hybridized with K11 RNA probes, after 3 minutes of ischemia and 4-48 hours of recovery. An upregulation of K11 was observed during recovery in regions of the brain sensitive to ischemia.

FIG. 3 depicts K11 expression in damaging and protecting ischemia. Rat brain coronal sections were hybridized with K11 RNA probes after 10 min of global ischemia with (pci) or without (nci) preconditioning and 12, 18, 24 hours of recovery (n=4). A higher degree of upregulation of K11 was detected in regions of the brain sensitive to ischemia in non condition compared to precondition animals. This demonstrates the involvement of K11 in pathological processes following cerebral ischemia.

FIG. 4 is a quantification of K11 mRNA expression after 2 hours of MCAO. Animals subjected to 2 h of MCAO were terminated at 2 hours of reperfusion and. RNA was extracted from both ischemic (ipsilateral) and non-ischemic (contralateral) regions. Real time PCR on these tissues shows an up-regulation of K11 by 12 fold in the ischemic region compare to non-ischemic region.

FIG. 5 is a graph depicting quantification of K11 mRNA expression after 90 minutes of oxygen/glucose deprivation (OGD)-induced cell death in primary cortical neurons. Cortical neurons were subjected to 90 min of OGD and 4-18 h. of recovery. K11 was up regulated by OGD in all time points. The upregulation indicates that K11 is induced before cell death.

FIG. 6 is a graph depicting the quantification and tissue distribution of K11 mRNA.

FIG. 7 is an autoradiogram of a northern blot using K11 as probe. It can be seen that there are two bands present in brain tissue, and a single band in testis.

FIG. 8 depicts a western blot of anti K11 antibodies, showing a band at about 105 KD for recombinantly expressed K11.

FIG. 9 depicts a western blot of brain tissue, where one of the K11 antibodies binds to an approximately 60 KD protein, and the other to a 30 KD protein, indicating that the K11 protein is cleaved in brain tissue.

FIG. 10 shows the immunohistochemistry of K11 expression in neurons, and co-localization with synaptophysin.

FIG. 11 shows immunohistochemistry of K11 expression after 90 minutes OGD, and recovery.

FIG. 12 shows immunohistochemistry of K11 expression in control brains and after 2 hours of MCAO and recovery.

### DETAILED DESCRIPTION OF THE EMBODIMENTS

Methods and compositions for the diagnosis and treatment of neuronal disease, including but not limited to neuronal ischemia, are described. The invention is based, in part, on the evaluation of the expression and role of genes that are differentially expressed in neural tissue after middle cerebral artery occlusion (MCAO). Neural tissue samples for expression analysis are taken from both the ischemic core and the ischemic penumbra at varying time points after occlusion and analyzed for differential expression of genes. Identification of these genes permits the definition of disease pathways, and the identification of targets in pathways that are useful both diagnostically and therapeutically.

Included are nucleic acid sequences of the novel ischemia associated gene, K11, provided as SEQ ID NO: 1 (rat) and SEQ ID NO: 3 (human), and the encoded polypeptides as SEQ ID NO: 2 and 4, respectively. Expression of K11 mRNA is upregulated shortly after ischemia in neuronal cells. Modulation of expression of K11 is useful in manipulating the ischemia state. K11 mRNA is expressed at low levels on control (non-ischemic) brain. It is also expressed in the testis. K11 is found to co-localize with synaptophysin, although it is also expressed in whole cells.

Also provided in the Seqlist is a variant sequence of the K11 gene (Genbank entry NM_{—}153626/NP_{—}795890; SEQ ID NO: 13. The variant sequence differs from SEQ ID NO: 1 at amino acid residue 30 (V30A) and at amino acid residue 280 (V280A). For some purposes that variant sequence may be used.

In response to ischemia, K11 is upregulated in the cell body, as well as dendrites of cortical and hippocampal neurons in regions of brain that are sensitive to ischemia, indicating involvement in cellular mechanism of ischemic neuronal injury. In normal brains, K11 protein is found in synapses and dendrites of the CA1 region of the hippocampus, indicating a role in synaptic transmission and memory formation. Targeting or modulating K11 and proteins regulated by K11 are of great interest for the development of therapeutic intervention against ischemic damage and the enhancement of memory function in the brain.

The mRNA and protein of K11 are altered by both focal and global ischemic insults prior to cell death in the affected regions of the brain. In focal ischemic model an up regulation is present during entire time of reperfusion, which correlates to the protein expression in the affected regions. In global model of ischemic insult, when a selective and delayed neuronal damage is present, there is a persistent up regulation prior to cell death.

K11 was identified by creating cDNA libraries from ischemic tissue, identifying differentially expressed clones by DNA sequencing, that were then printed on high-density arrays for determination of temporal gene expression profiles. Differential expression and expression patterns of induced genes were confirmed by in situ hybridization on tissue generated from ischemic models. Such comprehensive gene expression profiling in addition to functional analysis has elucidated mechanisms of ischemic neuronal damage, and identified drug target candidates for therapeutic intervention. Functional modulation of this gene and its product provides a point of intervention to block neuronal death after ischemia, and also provides therapeutic intervention in other central nervous system diseases with similar pathophysiology to stroke, including traumatic brain injury epilepsy and neurodegenerative diseases, e.g. Alzheimer's disease, Parkinson's disease, and the like.

The data indicate that the naturally occurring, functional K11 protein in brain tissue is cleaved into an approximately 60 KD fragment, and an approximately 30 KD fragment. Sequence analysis indicates that among the proteins containing HLH-2 domain and two PAS domains K11 is most closely related to C15C82_CAEEL Q963j4 C. elegans; a putative transcription factor c15c8.2; and cranky of D. melanogaster (NP_{—}651463). The sequence alignment of K11 and the C. elegans homolog clearly shows a termination at the position corresponding to residue 552 of SEQ ID NO: 2, and SEQ ID NO: 4. This suggests a domain boundary, and position for cleavage close to this site by a protease, thereby removing the C-terminal part. In one embodiment of the invention, a fragment of K11is provided that comprises residues 1-552 of SEQ ID NO: 2 or SEQ ID NO: 4. It will be understood by one of skill in the art that minor changes may occur in the cleavage site, e.g. residues 1-551, 1-550, 1-449, and smaller, e.g. 1-445, 1-440; or larger, 1-555, 1-560, and the like. In another embodiment, a fragment is provided that comprises residues 553-802. It will be understood by one of skill in the art that minor changes may occur in the cleavage site, e.g. residues 551-802, 550-802, 449-802, and larger, e.g. 445-802, 440-802; or smaller, 555-802, 560-802, and the like.

The protease that catalyzes this cleavage is also of interest. In one embodiment of the invention, compounds are screened for biological activity by determining the modulation of this cleavage, e.g. by determining the ability of an agent to inhibit the cleavage of the K11 gene product, which may be a recombinantly produced gene product. For example, a recombinantly produced K11 protein may be contacted with a brain tissue lysate in the absence or presence of a candidate agent, and the cleavage of K11 detected. Agents thus identified as affecting the cleavage are of interest in therapies directed at ischemia and stroke, as well as other neurodegenerative diseases like Alzheimer's disease and Parkinson's Disease.

The identification of K11 provides diagnostic and prognostic methods, which detect the occurrence of a neurologic disorder, e.g. ischemia or stroke, or assess an individual's susceptibility to such disease, by detecting upregulation of ischemia associated genes or by detecting the K11 in cerebral spinal fluid and blood for development of diagnostic kits for stroke. Therapeutic and prophylactic treatment methods for individuals suffering, or at risk of a neurological disorder such as stroke, involve administering either a therapeutic or prophylactic amount of an agent that modulates the activity of K11. Agents of interest include purified forms of the K11 protein, agents that stimulate expression or synthesis of such gene products, agents that block activity of such gene products or that down regulate the expression of such genes, or a nucleic acid, including coding sequences of K11 or anti-sense or RNAi sequences corresponding to K11.

Screening methods generally involve conducting various types of assays to identify agents that modulate the expression or activity of a K11 protein. Such screening methods can initially involve screens to identify compounds that can bind to the protein. Certain assays are designed to measure more clearly the effect that different agents have on neuroprotective or neuro-damaging gene product activities or expression levels. Lead compounds identified during these screens can serve as the basis for the synthesis of more active analogs. Lead compounds and/or active analogs generated therefrom can be formulated into pharmaceutical compositions effective in treating neurological disorders such as stroke, epilepsy and neurodegenerative disorders.

Disease Conditions

“Neurologic disorder” is defined here and in the claims as a disorder in which dysfunction and loss of neurons occurs either in the peripheral nervous system or in the central nervous system. Examples of neurologic disorders include: chronic neurodegenerative diseases such as Alzheimer's disease, Parkinson's disease, Huntington's chorea, diabetic peripheral neuropathy, multiple sclerosis, amyotrophic lateral sclerosis, aging, and acute disorders including: stroke, traumatic brain injury, peripheral nerve damage, hypoglycemia, spinal cord injury, epilepsy, and anoxia and hypoxia. Modulation of K11 activity also find use in the enhancement of normal memory formation and brain function.

The term “stroke” broadly refers to the development of neurological deficits associated with impaired blood flow to the brain regardless of cause. Potential causes include, but are not limited to, thrombosis, hemorrhage and embolism. Current methods for diagnosing stroke include symptom evaluation, medical history, chest X-ray, ECG (electrical heart activity), EEG (brain nerve cell activity), CAT scan to assess brain damage and MRI to obtain internal body visuals. Thrombus, embolus, and systemic hypotension are among the most common causes of cerebral ischemic episodes. Other injuries may be caused by hypertension, hypertensive cerebral vascular disease, rupture of an aneurysm, an angioma, blood dyscrasias, cardiac failure, cardic arrest, cardiogenic shock, septic shock, head trauma, spinal cord trauma, seizure, bleeding from a tumor, or other blood loss.

By “ischemic episode” is meant any circumstance that results in a deficient supply of blood to a tissue. When the ischemia is associated with a stroke, it can be either global or focal ischemia, as defined below. The term “ischemic stroke” refers more specifically to a type of stroke that is of limited extent and caused due to blockage of blood flow.

Cerebral ischemic episodes result from a deficiency in the blood supply to the brain. The spinal cord, which is also a part of the central nervous system, is equally susceptible to ischemia resulting from diminished blood flow.

By “focal ischemia,” as used herein in reference to the central nervous system, is meant the condition that results from the blockage of a single artery that supplies blood to the brain or spinal cord, resulting in damage to the cells in the territory supplied by that artery.

By “global ischemia,” as used herein in reference to the central nervous system, is meant the condition that results from a general diminution of blood flow to the entire brain, forebrain, or spinal cord, which causes the death of neurons in selectively vulnerable regions throughout these tissues. The pathology in each of these cases is quite different, as are the clinical correlates. Models of focal ischemia apply to patients with focal cerebral infarction, while models of global ischemia are analogous to cardiac arrest, and other causes of systemic hypotension.

Stroke can be modeled in animals, such as the rat (for a review see Duverger et al. (1988) J Cereb Blood Flow Metab 8(4):449-61), by occluding certain cerebral arteries that prevent blood from flowing into particular regions of the brain, then releasing the occlusion and permitting blood to flow back into that region of the brain (reperfusion). These focal ischemia models are in contrast to global ischemia models where blood flow to the entire brain is blocked for a period of time prior to reperfusion. Certain regions of the brain are particularly sensitive to this type of ischemic insult. The precise region of the brain that is directly affected is dictated by the location of the blockage and duration of ischemia prior to reperfusion. One model for focal cerebral ischemia uses middle cerebral artery occlusion (MCAO) in rats. Studies in normotensive rats can produce a standardized and repeatable infarction. MCAO in the rat mimics the increase in plasma catecholamines, electrocardiographic changes, sympathetic nerve discharge, and myocytolysis seen in the human patient population.

The methods of the invention are also useful for diagnosis and treatment of injuries to the central nervous system that are caused by mechanical forces, such as a blow to the head or spine. Trauma can involve a tissue insult such as an abrasion, incision, contusion, puncture, compression, etc., such as can arise from traumatic contact of a foreign object with any locus of or appurtenant to the head, neck, or vertebral column. Other forms of traumatic injury can arise from constriction or compression of CNS tissue by an inappropriate accumulation of fluid (for example, a blockade or dysfunction of normal cerebrospinal fluid or vitreous humor fluid production, turnover, or volume regulation, or a subdural or intracranial hematoma or edema). Similarly, traumatic constriction or compression can arise from the presence of a mass of abnormal tissue, such as a metastatic or primary tumor.

Identification of K11

In order to identify K11, tissue was taken at defined time points after MCAO from areas of the brain that are affected, e.g. the ischemic core or penumbra, or from unaffected tissue. Sham treated brain tissue was used as a control. RNA was isolated from one or more such tissues. Differentially expressed genes were detected by comparing the pattern of gene expression between the experimental and control conditions. Once a particular gene was identified, its expression pattern was further characterized by high-density arrays for determination of temporal gene expression profiles. Differential expression and expression pattern of induced genes were confirmed by in situ hybridization or RT-PCR on tissue generated from ischemic models.

“Differential expression” as used herein refers to both quantitative as well as qualitative differences in the genes' temporal and/or tissue expression patterns. Thus, a differentially expressed gene may have its expression activated or completely inactivated in normal versus neuronal disease conditions, or under control versus experimental conditions. Such a qualitatively regulated gene will exhibit an expression pattern within a given tissue or cell type that is detectable in either control or neuronal disease subjects, but is not detectable in both. Detectable, as used herein, refers to an RNA expression pattern that is detectable via the standard techniques of differential display, reverse transcriptase- (RT-) PCR and/or Northern analyses, which are well known to those of skill in the art. Generally, differential expression means that there is at least a 20% change, and in other instances at least a 2-, 3-, 5- or 10-fold difference between disease and control tissue expression. The difference usually is one that is statistically significant, meaning that the probability of the difference occurring by chance (the P-value) is less than some predetermined level (e.g., 0.05). Usually the confidence level P is <0.05, more typically <0.01, and in other instances, <0.001.

Alternatively, a differentially expressed gene may have its expression modulated, i.e., quantitatively increased or decreased, in normal versus neuronal disease states, or under control versus experimental conditions. The difference in expression need only is large enough to be visualized via standard detection techniques as described above.

Once a sequence has been identified as differentially expressed, the sequence can be subjected to a functional validation process to determine whether the gene plays a role in ischemia. Such candidate genes can potentially be correlated with a wide variety of cellular states or activities, including protective responses to an ischemic episode. The term “functional validation” as used herein refers to a process whereby one determines whether modulation of expression of a candidate gene or set of such genes causes a detectable change in a cellular activity or cellular state for a reference cell, which cell can be a population of cells such as a tissue or an entire organism. The detectable change or alteration that is detected can be any activity carried out by the reference cell. Specific examples of activities or states in which alterations can be detected include, but are not limited to, phenotypic changes (e.g., cell morphology, cell proliferation, cell viability and cell death); cells acquiring resistance to a prior sensitivity or acquiring a sensitivity which previously did not exist; protein/protein interactions; cell movement; intracellular or intercellular signaling; cell/cell interactions; cell activation (e.g., T cell activation, B cell activation, mast cell degranulation); release of cellular components (e.g., hormones, chemokines and the like); and metabolic or catabolic reactions.

A variety of options are available for functionally validating candidate genes. For example, a number of options are available to detect interference of candidate gene expression (i.e., to detect candidate gene silencing). In general, inhibition in expression is detected by detecting a decrease in the level of the protein encoded by the candidate gene, determining the level of mRNA transcribed from the gene and/or detecting a change in phenotype associated with candidate gene expression.

Such methods as RNAi technology can be used. Antisense technology can also be utilized to functionally validate a candidate gene. In this approach, an antisense polynucleotide that specifically hybridizes to a segment of the coding sequence for the candidate gene is administered to inhibit expression of the candidate gene in those cells into which it is introduced. The functional role that a candidate gene plays in a cell can also be assessed using gene “knockout” approaches in which the candidate gene is deleted, modified, or inhibited on either a single or both alleles. The cells or animals can be optionally be reconstituted with a wild-type candidate gene as part of a further analysis.

In one embodiment of the invention, RNAi technology is used in functional validation. As used herein, RNAi technology refers to a process in which double-stranded RNA is introduced into cells expressing a candidate gene to inhibit expression of the candidate gene, i.e., to “silence” its expression. The dsRNA is selected to have substantial identity with the candidate gene. In general such methods initially involve transcribing a nucleic acids containing all or part of a candidate gene into single- or double-stranded RNA. Sense and anti-sense RNA strands are allowed to anneal under appropriate conditions to form dsRNA. The resulting dsRNA is introduced into reference cells via various methods and the degree of attenuation in expression of the candidate gene is measured using various techniques. Usually one detects whether inhibition alters a cellular state or cellular activity.

Nucleic Acids

The coding sequence of the rat K11 is set forth in SEQ ID NO: 1, and the encoded polypeptide in SEQ ID NO: 2. The human counterparts are provided as SEQ ID NO: 3 and SEQ ID NO: 4, respectively. Nucleic acids comprising K11 sequences find use in diagnostic and prognostic methods, for the recombinant production of the encoded polypeptide, and the like. The nucleic acids of the invention include nucleic acids having a high degree of sequence similarity or sequence identity to SEQ ID NO: 1. Sequence identity can be determined by hybridization under stringent conditions, for example, at 50° C. or higher and 0.1×SSC (9 mM NaCl/0.9 mM Na citrate). Hybridization methods and conditions are well known in the art, see, e.g., U.S. Pat. No. 5,707,829. Nucleic acids that are substantially identical to the provided nucleic acid sequence, e.g. allelic variants, genetically altered versions of the gene, etc., bind to SEQ ID NO: 1. Further specific guidance regarding the preparation of nucleic acids is provided by Fleury et al. (1997) Nature Genetics 15:269-272; Tartaglia et al., PCT Publication No. WO 96/05861; and Chen et al., PCT Publication No. WO 00/06087, each of which is incorporated herein in its entirety.

The K11 gene may be obtained using various methods well known to those skilled in the art, including but not limited to the use of appropriate probes to detect the gene within an appropriate cDNA or genomic DNA library, antibody screening of expression libraries to detect cloned DNA fragments with shared structural features, direct chemical synthesis, and amplification protocols. Libraries are preferably prepared from neural cells. Cloning methods are described in Berger and Kimmel, Guide to Molecular Cloning Techniques, Methods in Enzymology, 152, Academic Press, Inc. San Diego, Calif.; Sambrook, et al. (1989) Molecular Cloning—A Laboratory Manual (2nd ed) Vols. 1-3, Cold Spring Harbor Laboratory, Cold Spring Harbor Press, N.Y.; and Current Protocols (1994), a joint venture between Greene Publishing Associates, Inc. and John Wiley and Sons, Inc.

The sequence obtained from partial K11 clones can be used to obtain the entire coding region by using the RACE method (Chenchik et al. (1995) CLONTECHniques (X) 1: 5-8). Oligonucleotides can be designed based on the sequence obtained from the partial clone that can amplify a reverse transcribed mRNA encoding the entire coding sequence. Alternatively, probes can be used to screen cDNA libraries prepared from an appropriate cell or cell line in which the gene is transcribed. Once the target nucleic acid is identified, it can be isolated and cloned using well-known amplification techniques. Such techniques include the polymerase chain reaction (PCR) the ligase chain reaction (LCR), Qβ-replicase amplification, the self-sustained sequence replication system (SSR) and the transcription based amplification system (TAS). Such methods include, those described, for example, in U.S. Pat. No. 4,683,202 to Mullis et al.; PCR Protocols A Guide to Methods and Applications (Innis et al. eds) Academic Press Inc. San Diego, Calif. (1990); Kwoh et al. (1989) Proc. Natl. Acad. Sci. USA 86: 1173; Guatelli et al. (1990) Proc. Natl. Acad. Sci. USA 87: 1874; Lomell et al. (1989) J. Clin. Chem. 35: 1826; Landegren et al. (1988) Science 241: 1077-1080; Van Brunt (1990) Biotechnology 8: 291-294; Wu and Wallace (1989) Gene 4: 560; and Barringer et al. (1990) Gene 89: 117.

As an alternative to cloning a nucleic acid, a suitable nucleic acid can be chemically synthesized. Direct chemical synthesis methods include, for example, the phosphotriester method of Narang et al. (1979) Meth. Enzymol. 68: 90-99; the phosphodiester method of Brown et al. (1979) Meth. Enzymol. 68: 109-151; the diethylphosphoramidite method of Beaucage et al. (1981) Tetra. Lett, 22: 1859-1862; and the solid support method of U.S. Pat. No. 4,458,066. Chemical synthesis produces a single stranded oligonucleotide. This can be converted into double stranded DNA by hybridization with a complementary sequence, or by polymerization with a DNA polymerase using the single strand as a template. While chemical synthesis of DNA is often limited to sequences of about 100 bases, longer sequences can be obtained by the ligation of shorter sequences. Alternatively, subsequences may be cloned and the appropriate subsequences cleaved using appropriate restriction enzymes.

K11 nucleic acids can be cDNAs or genomic DNAs, as well as fragments thereof. The term “cDNA” as used herein is intended to include all nucleic acids that share the arrangement of sequence elements found in native mature mRNA species, where sequence elements are exons and 3′ and 5′ non-coding regions. Normally mRNA species have contiguous exons, with the intervening introns, when present, being removed by nuclear RNA splicing, to create a continuous open reading frame encoding a polypeptide of the invention.

A genomic sequence of interest comprises the nucleic acid present between the initiation codon and the stop codon, as defined in the listed sequences, including all of the introns that are normally present in a native chromosome. It can further include the 3′ and 5′ untranslated regions found in the mature mRNA. It can further include specific transcriptional and translational regulatory sequences, such as promoters, enhancers, etc., including about 1 kb, but possibly more, of flanking genomic DNA at either the 5′ or 3′ end of the transcribed region. The genomic DNA flanking the coding region, either 3′ or 5′, or internal regulatory sequences as sometimes found in introns, contains sequences required for proper tissue, stage-specific, or disease-state specific expression, and are useful for investigating the up-regulation of expression in tumor cells.

Probes specific to K11 can be generated using the nucleic acid sequence disclosed in SEQ ID NO: 1 or SEQ ID NO: 3. The probes are preferably at least about 18 nt, 25 nt, 50 nt or more of the corresponding contiguous sequence of one of the sequences provided in SEQ ID NO: 1, and are usually less than about 500 bp in length. Preferably, probes are designed based on a contiguous sequence that remains unmasked following application of a masking program for masking low complexity, e.g. BLASTX. Double or single stranded fragments can be obtained from the DNA sequence by chemically synthesizing oligonucleotides in accordance with conventional methods, by restriction enzyme digestion, by PCR amplification, etc. The probes can be labeled, for example, with a radioactive, biotinylated, or fluorescent tag.

The nucleic acids of the subject invention are isolated and obtained in substantial purity, generally as other than an intact chromosome. Usually, the nucleic acids, either as DNA or RNA, will be obtained substantially free of other naturally-occurring nucleic acid sequences, generally being at least about 50%, usually at least about 90% pure and are typically “recombinant,” e.g., flanked by one or more nucleotides with which it is not normally associated on a naturally occurring chromosome.

The nucleic acids of the invention can be provided as a linear molecule or within a circular molecule, and can be provided within autonomously replicating molecules (vectors) or within molecules without replication sequences. Expression of the nucleic acids can be regulated by their own or by other regulatory sequences known in the art. The nucleic acids of the invention can be introduced into suitable host cells using a variety of techniques available in the art, such as transferrin polycation-mediated DNA transfer, transfection with naked or encapsulated nucleic acids, liposome-mediated DNA transfer, intracellular transportation of DNA-coated latex beads, protoplast fusion, viral infection, electroporation, gene gun, calcium phosphate-mediated transfection, and the like.

For use in amplification reactions, such as PCR, a pair of primers will be used. The exact composition of the primer sequences is not critical to the invention, but for most applications the primers will hybridize to the subject sequence under stringent conditions, as known in the art. It is preferable to choose a pair of primers that will generate an amplification product of at least about 50 nt, preferably at least about 100 nt. Algorithms for the selection of primer sequences are generally known, and are available in commercial software packages. Amplification primers hybridize to complementary strands of DNA, and will prime towards each other. For hybridization probes, it may be desirable to use nucleic acid analogs, in order to improve the stability and binding affinity. The term “nucleic acid” shall be understood to encompass such analogs.

Polypeptides

K11 polypeptides are of interest for screening methods, as reagents to raise antibodies, as therapeutics, and the like. Such polypeptides can be produced through isolation from natural sources, recombinant methods and chemical synthesis. In addition, functionally equivalent polypeptides may find use, where the equivalent polypeptide may contain deletions, additions or substitutions of amino acid residues that result in a silent change, thus producing a functionally equivalent differentially expressed on pathway gene product. Amino acid substitutions may be made on the basis of similarity in polarity, charge, solubility, hydrophobicity, hydrophilicity, and/or the amphipathic nature of the residues involved. “Functionally equivalent”, as used herein, refers to a protein capable of exhibiting a substantially similar in vivo activity as the polypeptide encoded by SEQ ID NO: 1 or SEQ ID NO: 3.

The polypeptides may be produced by recombinant DNA technology using techniques well known in the art. Methods that are well known to those skilled in the art can be used to construct expression vectors containing coding sequences and appropriate transcriptional/translational control signals. These methods include, for example, in vitro recombinant DNA techniques, synthetic techniques and in vivo recombination/genetic recombination. Alternatively, RNA capable of encoding the polypeptides of interest may be chemically synthesized.

Typically, the coding sequence is placed under the control of a promoter that is functional in the desired host cell to produce relatively large quantities of the gene product. An extremely wide variety of promoters are well known, and can be used in the expression vectors of the invention, depending on the particular application. Ordinarily, the promoter selected depends upon the cell in which the promoter is to be active. Other expression control sequences such as ribosome binding sites, transcription termination sites and the like are also optionally included. Constructs that include one or more of this control sequences are termed “expression cassettes.” Expression can be achieved in prokaryotic and eukaryotic cells utilizing promoters and other regulatory agents appropriate for the particular host cell. Exemplary host cells include, but are not limited to, E. coli, other bacterial hosts, yeast, and various higher eukaryotic cells such as the COS, CHO and HeLa cells lines and myeloma cell lines.

In mammalian host cells, a number of viral-based expression systems may be used, including retrovirus, lentivirus, adenovirus, adeno-associated virus, and the like. In cases where an adenovirus is used as an expression vector, the coding sequence of interest can be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing K11 protein in infected hosts.

Specific initiation signals may also be required for efficient translation of the genes. These signals include the ATG initiation codon and adjacent sequences. In cases where a complete gene, including its own initiation codon and adjacent sequences, is inserted into the appropriate expression vector, no additional translational control signals may be needed. However, in cases where only a portion of the gene coding sequence is inserted, exogenous translational control signals must be provided. These exogenous translational control signals and initiation codons can be of a variety of origins, both natural and synthetic. The efficiency of expression may be enhanced by the inclusion of appropriate transcription enhancer elements, transcription terminators, etc.

In addition, a host cell strain may be chosen that modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product may be used. Such mammalian host cells include but are not limited to CHO, VERO, BHK, HeLa, COS, MDCK, 293, 3T3, W138, etc.

For long-term, production of recombinant proteins, stable expression is preferred. For example, cell lines that stably express K11 may be engineered. Rather than using expression vectors that contain viral origins of replication, host cells can be transformed with DNA controlled by appropriate expression control elements, and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci, which in turn can be cloned and expanded into cell lines. This method may advantageously be used to engineer cell lines that express the target protein. Such engineered cell lines may be particularly useful in screening and evaluation of compounds that affect the endogenous activity of K11 protein. A number of selection systems may be used, including but not limited to the herpes simplex virus thymidine kinase, hypoxanthine-guanine phosphoribosyltransferase, and adenine phosphoribosyltransferase genes. Antimetabolite resistance can be used as the basis of selection for dhfr, which confers resistance to methotrexate; gpt, which confers resistance to mycophenolic acid; neo, which confers resistance to the aminoglycoside G-418; and hygro, which confers resistance to hygromycin.

The polypeptide may be labeled, either directly or indirectly. Any of a variety of suitable labeling systems may be used, including but not limited to, radioisotopes such as ¹²⁵I; enzyme labeling systems that generate a detectable colorimetric signal or light when exposed to substrate; and fluorescent labels. Indirect labeling involves the use of a protein, such as a labeled antibody, that specifically binds to the polypeptide of interest. Such antibodies include but are not limited to polyclonal, monoclonal, chimeric, single chain, Fab fragments and fragments produced by a Fab expression library.

Once expressed, the recombinant polypeptides can be purified according to standard procedures of the art, including ammonium sulfate precipitation, affinity columns, ion exchange and/or size exclusivity chromatography, gel electrophoresis and the like (see, generally, R. Scopes, Protein Purification, Springer-Overflag, N.Y. (1982), Deutsche, Methods in Enzymology Vol. 182: Guide to Protein Purification., Academic Press, Inc. N.Y. (1990)).

As an option to recombinant methods, polypeptides and oligopeptides can be chemically synthesized. Such methods typically include solid-state approaches, but can also utilize solution based chemistries and combinations or combinations of solid-state and solution approaches. Examples of solid-state methodologies for synthesizing proteins are described by Merrifield (1964) J. Am. Chem. Soc. 85:2149; and Houghton (1985) Proc. Natl. Acad. Sci., 82:5132. Fragments of an ischemia-associated protein can be synthesized and then joined together. Methods for conducting such reactions are described by Grant (1992) Synthetic Peptides: A User Guide, W.H. Freeman and Co., N.Y.; and in “Principles of Peptide Synthesis,” (Bodansky and Trost, ed.), Springer-Verlag, Inc. N.Y., (1993).

Specific Binding Members

The term “specific binding member” or “binding member” as used herein refers to a member of a specific binding pair, i.e. two molecules, usually two different molecules, where one of the molecules (i.e., first specific binding member) through chemical or physical means specifically binds to the other molecule (i.e., second specific binding member). The complementary members of a specific binding pair are sometimes referred to as a ligand and receptor; or receptor and counter-receptor. For the purposes of the present invention, the two binding members may be known to associate with each other, for example where an assay is directed at detecting compounds that interfere with the association of a known binding pair. Alternatively, candidate compounds suspected of being a binding partner to a compound of interest may be used.

Specific binding pairs of interest include carbohydrates and lectins; complementary nucleotide sequences; peptide ligands and receptor; effector and receptor molecules; hormones and hormone binding protein; enzyme cofactors and enzymes; enzyme inhibitors and enzymes; lipid and lipid-binding protein; etc. The specific binding pairs may include analogs, derivatives and fragments of the original specific binding member. For example, a receptor and ligand pair may include peptide fragments, chemically synthesized peptidomimetics, labeled protein, derivatized protein, etc.

In a preferred embodiment, the specific binding member is an antibody. The term “antibody” or “antibody moiety” is intended to include any polypeptide chain-containing molecular structure with a specific shape that fits to and recognizes an epitope, where one or more non-covalent binding interactions stabilize the complex between the molecular structure and the epitope. The term includes monoclonal antibodies, multispecific antibodies (antibodies that include more than one domain specificity), human antibody, humanized antibody, and antibody fragments with the desired biological activity.

Antibodies that bind specifically to K11 are referred to as anti-K11 antibodies. The specific or selective fit of a given structure and its specific epitope is sometimes referred to as a “lock and key” fit. The archetypal antibody molecule is the immunoglobulin, and all types of immunoglobulins, IgG, e.g. IgG1, IgG2a, IgG2b, IgG3, IgG4, IgM, IgA, IgE, IgD, etc., from all sources, e.g. human, rodent, rabbit, cow, sheep, pig, dog, other mammal, chicken, other avians, etc., are considered to be “antibodies.” Antibodies utilized in the present invention may be polyclonal antibodies, although monoclonal antibodies are preferred because they may be reproduced by cell culture or recombinantly, and can be modified to reduce their antigenicity.

Polyclonal antibodies can be raised by a standard protocol by injecting a production animal with an antigenic composition, formulated as described above, and as described in the examples. See, e.g., Harlow and Lane, Antibodies: A Laboratory Manual, Cold Spring Harbor Laboratory, 1988. In one such technique, a K11 antigen comprising an antigenic portion of the polypeptide is initially injected into any of a wide variety of mammals (e.g., mice, rats, rabbits, sheep or goats). The K11 antigen may comprise whole protein, or may be specific for the C terminal or N terminal cleavage products.

When utilizing an entire protein, or a larger section of the protein, antibodies may be raised by immunizing the production animal with the protein and a suitable adjuvant (e.g., Fruend's, Fruend's complete, oil-in-water emulsions, etc.) When a smaller peptide is utilized, it is advantageous to conjugate the peptide with a larger molecule to make an immunostimulatory conjugate. Commonly utilized conjugate proteins that are commercially available for such use include bovine serum albumin (BSA) and keyhole limpet hemocyanin (KLH). In order to raise antibodies to particular epitopes, peptides derived from the full sequence may be utilized. Alternatively, in order to generate antibodies to relatively short peptide portions of the brain tumor protein target, a superior immune response may be elicited if the polypeptide is joined to a carrier protein, such as ovalbumin, BSA or KLH. The peptide-conjugate is injected into the animal host, preferably according to a predetermined schedule incorporating one or more booster immunizations, and the animals are bled periodically. Polyclonal antibodies specific for the polypeptide may then be purified from such antisera by, for example, affinity chromatography using the polypeptide coupled to a suitable solid support.

Alternatively, for monoclonal antibodies, hybridomas may be formed by isolating the stimulated immune cells, such as those from the spleen of the inoculated animal. These cells are then fused to immortalized cells, such as myeloma cells or transformed cells, which are capable of replicating indefinitely in cell culture, thereby producing an immortal, immunoglobulin-secreting cell line. The immortal cell line utilized is preferably selected to be deficient in enzymes necessary for the utilization of certain nutrients. Many such cell lines (such as myelomas) are known to those skilled in the art, and include, for example: thymidine kinase (TK) or hypoxanthine-guanine phosphoriboxyl transferase (HGPRT). These deficiencies allow selection for fused cells according to their ability to grow on, for example, hypoxanthine aminopterinthymidine medium (HAT).

Preferably, the immortal fusion partners utilized are derived from a line that does not secrete immunoglobulin. The resulting fused cells, or hybridomas, are cultured under conditions that allow for the survival of fused, but not unfused, cells and the resulting colonies screened for the production of the desired monoclonal antibodies. Colonies producing such antibodies are cloned, expanded, and grown so as to produce large quantities of antibody, see Kohler and Milstein, 1975 Nature 256:495 (the disclosures of which are hereby incorporated by reference).

Large quantities of monoclonal antibodies from the secreting hybridomas may then be produced by injecting the clones into the peritoneal cavity of mice and harvesting the ascites fluid therefrom. The mice, preferably primed with pristane, or some other tumor-promoter, and immunosuppressed chemically or by irradiation, may be any of various suitable strains known to those in the art. The ascites fluid is harvested from the mice and the monoclonal antibody purified therefrom, for example, by CM Sepharose column or other chromatographic means. Alternatively, the hybridomas may be cultured in vitro or as suspension cultures. Batch, continuous culture, or other suitable culture processes may be utilized. Monoclonal antibodies are then recovered from the culture medium or supernatant.

In addition, the antibodies or antigen binding fragments may be produced by genetic engineering. In this technique, as with the standard hybridoma procedure, antibody-producing cells are sensitized to the desired antigen or immunogen. The messenger RNA isolated from the immune spleen cells or hybridomas is used as a template to make cDNA using PCR amplification. A library of vectors, each containing one heavy chain gene and one light chain gene retaining the initial antigen specificity, is produced by insertion of appropriate sections of the amplified immunoglobulin cDNA into the expression vectors. A combinatorial library is constructed by combining the heavy chain gene library with the light chain gene library. This results in a library of clones, which co-express a heavy and light chain (resembling the Fab fragment or antigen binding fragment of an antibody molecule). The vectors that carry these genes are co-transfected into a host (e.g. bacteria, insect cells, mammalian cells, or other suitable protein production host cell.). When antibody gene synthesis is induced in the transfected host, the heavy and light chain proteins self-assemble to produce active antibodies that can be detected by screening with the antigen or immunogen.

Preferably, recombinant antibodies are produced in a recombinant protein production system that correctly glycosylates and processes the immunoglobulin chains, such as insect or mammalian cells. An advantage to using insect cells, which utilize recombinant baculoviruses for the production of antibodies, is that the baculovirus system allows production of mutant antibodies much more rapidly than stably transfected mammalian cell lines. In addition, insect cells have been shown to correctly process and glycosylate eukaryotic proteins, which prokaryotic cells do not. Finally, the baculovirus expression of foreign protein has been shown to constitute as much as 50-75% of the total cellular protein late in viral infection, making this system an excellent means of producing milligram quantities of the recombinant antibodies.

Antibodies with a reduced propensity to induce a violent or detrimental immune response in humans (such as anaphylactic shock), and which also exhibit a reduced propensity for priming an immune response which would prevent repeated dosage with the antibody therapeutic or imaging agent are preferred for use in the invention. Even through the brain is relatively isolated behind the blood brain barrier, an immune response still can occur in the form of increased leukocyte infiltration, and inflammation. Thus, humanized, single chain, chimeric, or human antibodies, which produce less of an immune response when administered to humans, are preferred for use in the present invention. Also included in the invention are multi-domain antibodies, and anti-idiotypic antibodies that “mimic” K11. Such anti-idiotypic antibodies or Fab fragments of such anti-idiotypes can be used in therapeutic regimens involving a K11 mediated pathway (see, for example, Greenspan and Bona (1993) FASEB J 7(5):437-444; Nissinoff (1991) J. Immunol. 147(8):2429-2438.

A chimeric antibody is a molecule in which different portions are derived from different animal species, for example those having a variable region derived from a murine mAb and a human immunoglobulin constant region. Techniques for the development of chimeric antibodies are described in the literature. See, for example, Morrison et al. (1984) Proc. Natl. Acad. Sci. 81:6851-6855; Neuberger et al. (1984) Nature 312:604-608; Takeda et al. (1985) Nature 314:452-454. Single chain antibodies are formed by linking the heavy and light chain fragments of the Fv region via an amino acid bridge, resulting in a single chain polypeptide. See, for example, Huston et al., Science 242:423-426; Proc. Natl. Acad. Sci. 85:5879-5883; and Ward et al. Nature 341:544-546.

Antibody fragments that recognize specific epitopes may be generated by techniques well known in the field. These fragments include, without limitation, F(ab′)₂ fragments, which can be produced by pepsin digestion of the antibody molecule, and Fab fragments, which can be generated by reducing the disulfide bridges of the F(ab′)₂ fragments.

Humanized antibodies are human forms of non-human antibodies. They are chimeras with a minimum sequence derived from of non-human Immunoglobulin. To overcome the intrinsic undesirable properties of murine monoclonal antibodies, recombinant murine antibodies engineered to incorporate regions of human antibodies, also called “humanized antibodies” are being developed. This alternative strategy may be adopted as it is difficult to generate human antibodies directed to human antigens. A humanized antibody contains complementarity determining region (CDR) regions and a few other amino acid of a murine antibody while the rest of the antibody is of human origin.

Chimeric antibodies may be made by recombinant means by combining the murine variable light and heavy chain regions (VK and VH), obtained from a murine,(or other animal-derived) hybridoma clone, with the human constant light and heavy chain regions, in order to produce an antibody with predominantly human domains. The production of such chimeric antibodies is well known in the art, and may be achieved by standard means (as described, e.g., in U.S. Pat. No. 5,624,659, incorporated fully herein by reference). Humanized antibodies are engineered to contain even more human-like immunoglobulin domains, and incorporate only the complementarity-determining regions of the animal-derived antibody. This is accomplished by carefully examining the sequence of the hyper-variable loops of the variable regions of the monoclonal antibody, and fitting them to the structure of the human antibody chains. Although facially complex, the process is straightforward in practice. See, e.g., U.S. Pat. No. 6,187,287, incorporated fully herein by reference.

Alternatively, polyclonal or monoclonal antibodies may be produced from animals that have been genetically altered to produce human immunoglobulins. Techniques for generating such animals, and deriving antibodies therefrom, are described in U.S. Pat. Nos. 6,162,963 and 6,150,584, incorporated fully herein by reference.

In addition to entire immunoglobulins (or their recombinant counterparts), immunoglobulin fragments comprising the epitope binding site (e.g., Fab′, F(ab′)₂, or other fragments) are useful as antibody moieties in the present invention. Such antibody fragments may be generated from whole immunoglobulins by ficin, pepsin, papain, or other protease cleavage. “Fragment,” or minimal immunoglobulins may be designed utilizing recombinant immunoglobulin techniques. For instance “Fv” immunoglobulins for use in the present invention may be produced by linking a variable light chain region to a variable heavy chain region via a peptide linker (e.g., poly-glycine or another sequence which does not form an alpha helix or beta sheet motif).

Fv fragments are heterodimers of the variable heavy chain domain (V_{H}) and the variable light chain domain (V_{L}). The heterodimers of heavy and light chain domains that occur in whole IgG, for example, are connected by a disulfide bond. Recombinant Fvs in which V_{H} and V_{L} are connected by a peptide linker are typically stable, see, for example, Huston et al., Proc. Natl. Acad, Sci. USA 85:5879-5883 (1988) and Bird et al., Science 242:423-426 (1988), both fully incorporated herein, by reference. Improved Fv's have been also been made which comprise stabilizing disulfide bonds between the V_{H} and V_{L} regions, as described in U.S. Pat. No. 6,147,203, incorporated fully herein by reference. Any of these minimal antibodies may be utilized in the present invention, and those which are humanized to avoid HAMA reactions are preferred for use in embodiments of the invention.

In addition, derivatized immunoglobulins with added chemical linkers, detectable moieties, such as fluorescent dyes, enzymes, substrates, chemiluminescent moieties and the like, or specific binding moieties, such as streptavidin, avidin, or biotin, and the like may be utilized in the methods and compositions of the present invention. For convenience, the term “antibody” or “antibody moiety” will be used throughout to generally refer to molecules which specifically bind to an epitope of the brain tumor protein targets, although the term will encompass all immunoglobulins, derivatives, fragments, recombinant or engineered immunoglobulins, and modified immunoglobulins, as described above.

Candidate anti-K11 antibodies can be tested for by any suitable standard means, e.g. ELISA assays, etc. As a first screen, the antibodies may be tested for binding against the immunogen, or against the entire brain tumor protein target extracellular domain or protein. As a second screen, anti-K11 candidates may be tested for binding to an appropriate tumor cell line, or to primary tumor tissue samples. For these screens, the anti-K11 candidate antibody may be labeled for detection. After selective binding to the brain tumor protein target is established, the candidate antibody, or an antibody conjugate produced as described below, may be tested for appropriate activity in an in vivo model, such as an appropriate tumor cell line, or in a mouse or rat human model, as described below. Methods include, but are not limited to, methods that measure binding affinity to a target, biodistribution of the compound within an animal or cell, or compound mediated cytotoxicity. These and other screening methods known in the art provide information on the ability of a compound to bind to, modulate, or otherwise interact with the specified target and are a measure of the compound's efficacy.

The binding affinity of the K11 antibody may be determined using Biacore SPR technology, as is known in the art. In this method, a first molecule is coupled to a Dextran CM-5 sensor chip (Pharmacia), and the bound molecule is used to capture the antibody being tested. The antigen is then applied at a specific flow rate, and buffer applied at the same flow rate, so that dissociation occurs. The association rate and dissociation rates and corresponding rate constants are determined by using BIA evaluation software. For example, see Malmqvist (1993) Surface plasmon resonance for detection and measurement of antibody-antigen affinity and kinetics. Volume: 5:282-286; and Davies (1994) Nanobiology 3:5-16. Sequential introduction of antibodies permits epitope mapping. Once the antigen has been introduced, the ability of a second antibody to bind to the antigen can be tested. Each reactant can be monitored individually in the consecutive formation of multimolecular complexes, permitting multi-site binding experiments to be performed.

Diagnostic and Prognostic Methods

The differential expression of K11 in response to an ischemic event indicates that it can serve as a marker for diagnosing individuals that have suffered a stroke. Diagnostic methods include detection of specific markers correlated with specific stages in the pathological processes leading to cell death following a stroke of damaging insult. Knowledge of the progression stage can be the basis for more accurate assessment of the most appropriate treatment and most appropriate administration of therapeutics following a stroke. Prognostic methods can also be utilized to monitor an individual's health status prior to and after a stroke, as well as in the assessment of the severity of the stroke and the likelihood and extent of recovery.

In general, such diagnostic and prognostic methods involve detecting an elevated level of expression of K11 transcripts or gene product in the cells or tissue of an individual or a sample therefrom. A variety of different assays can be utilized to detect an increase in K11 expression, including methods that detect gene transcript or protein levels. More specifically, the diagnostic and prognostic methods disclosed herein involve obtaining a sample from an individual and determining at least qualitatively, and preferably quantitatively, the level of a K11 expression in the sample. Usually this determined value or test value is compared against some type of reference or baseline value.

Nucleic acids or binding members such as antibodies that are specific for K11 polypeptide are used to screen patient samples for increased expression of the corresponding mRNA or protein, or for the presence of amplified DNA in the cell. Samples can be obtained from a variety of sources. For example, since the methods are designed primarily to diagnose and assess risk factors for humans to neurological disorders such as stroke, samples are typically obtained from a human subject. However, the methods can also be utilized with samples obtained from various other mammals, such as primates, e.g. apes and chimpanzees, mice, cats, rats, and other animals. Such samples are referred to as a patient sample.

Samples can be obtained from the tissues or fluids of an individual, as well as from cell cultures or tissue homogenates. For example, samples can be obtained from whole blood, serum, semen, saliva, tears, urine, fecal material, sweat, buccal, skin, spinal fluid and amniotic fluid. Also included in the term are derivatives and fractions of such cells and fluids. Samples can also be derived from in vitro cell cultures, including the growth medium, recombinant cells and cell components. The cells may be dissociated, in the case of solid tissues, or tissue sections may be analyzed. Alternatively a lysate of the cells may be prepared.

Because certain diagnostic methods involve evaluating the level of expression in brain cells, the sample can be obtained from various types of cells including, but not limited to, neurons, astrocytes and microglial cells. Current evidence indicates that one consequence of stroke is that the blood/brain barrier becomes more permeable. Stroke also results in the death of certain cells which, upon dying, are lysed, thus expelling cellular components. These components can then traverse the blood/brain barrier and be picked up by the circulatory system. Consequently, certain diagnostic and prognostic methods are conducted with blood samples.

Diagnostic samples are collected any time after an individual is suspected to have had a stroke, or has exhibited symptoms that predict stroke. It is preferable to minimize the period of time between a suspected stroke and taking of the sample, usually within at least about one week after a suspected stroke, more usually within at least about 3 days of a suspected stroke, and preferably within at least about 24 hours of a suspected stroke, more preferable within at least about 12 hours. In prophylactic testing, samples can be obtained from an individual who present with risk factors that indicate a susceptibility to stroke, which risk factors include high blood pressure, obesity, diabetes, etc. as part of a routine assessment of the individual's health status.

The various test values determined for a sample from an individual believed to have suffered a stroke or to be susceptible to stroke typically are compared against a baseline value to assess the extent of increased expression, if any. This baseline value can be any of a number of different values. In some instances, the baseline value is a value established in a trial using a healthy cell or tissue sample that is run in parallel with the test sample. Alternatively, the baseline value can be a statistical value (e.g., a mean or average) established from a population of control cells or individuals. For example, the baseline value can be a value or range which is characteristic of a control individual or control population. For instance, the baseline value can be a statistical value or range that is reflective of expression levels for the general population, or more specifically, healthy individuals not susceptible to stroke. Individuals not susceptible to stroke generally refer to those having no apparent risk factors correlated with stroke, such as high blood pressure, high cholesterol levels, diabetes, smoking and high salt diet, for example.

Nucleic Acid Screening Methods

Some of the diagnostic and prognostic methods that involve the detection of a K11 transcript begin with the lysis of cells and subsequent purification of nucleic acids from other cellular material, particularly mRNA transcripts. A nucleic acid derived from an mRNA transcript refers to a nucleic acid for whose synthesis the mRNA transcript, or a subsequence thereof, has ultimately served as a template. Thus, a cDNA reverse transcribed from an mRNA, an RNA transcribed from that cDNA, a DNA amplified from the cDNA, an RNA transcribed from the amplified DNA, are all derived from the mRNA transcript and detection of such derived products is indicative of the presence and/or abundance of the original transcript in a sample. Thus, suitable samples include, but are not limited to, mRNA transcripts of K11, cDNA reverse transcribed from the mRNA, cRNA transcribed from the cDNA, DNA amplified from K11 nucleic acids, and RNA transcribed from amplified DNA.

A number of methods are available for analyzing nucleic acids for the presence of a specific sequence, e.g. upregulated expression. The nucleic acid may be amplified by conventional techniques, such as the polymerase chain reaction (PCR), to provide sufficient amounts for analysis. The use of the polymerase chain reaction is described in Saiki et al. (1985) Science 239:487, and a review of techniques may be found in Sambrook, et al. Molecular Cloning: A Laboratory Manual, CSH Press 1989, pp.14.2-14.33.

A detectable label may be included in an amplification reaction. Suitable labels include fluorochromes, e.g. fluorescein isothiocyanate (FITC), rhodamine, Texas Red, phycoerythrin, allophycocyanin,6-carboxyfluorescein(6-FAM),2,7-dimethoxy-4,5-dichloro-6-carboxyfluorescein (JOE), 6-carboxy-X-rhodamine (ROX), 6-carboxy-2,4,7,4,7-hexachlorofluorescein (HEX), 5-carboxyfluorescein (5-FAM) or N,N,N,N-tetramethyl-6-carboxyrhodamine (TAMRA), radioactive labels, e.g. ³²p, 35S, ³H; etc. The label may be a two stage system, where the amplified DNA is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

The sample nucleic acid, e.g. amplified, labeled, cloned fragment, etc. is analyzed by one of a number of methods known in the art. Probes may be hybridized to northern or dot blots, or liquid hybridization reactions performed. The nucleic acid may be sequenced by dideoxy or other methods, and the sequence of bases compared to a wild-type sequence. Single strand conformational polymorphism (SSCP) analysis, denaturing gradient gel electrophoresis (DGGE), and heteroduplex analysis in gel matrices are used to detect conformational changes created by DNA sequence variation as alterations in electrophoretic mobility. Fractionation is performed by gel or capillary electrophoresis, particularly acrylamide or agarose gels.

In situ hybridization methods are hybridization methods in which the cells are not lysed prior to hybridization. Because the method is performed in situ, it has the advantage that it is not necessary to prepare RNA from the cells. The method usually involves initially fixing test cells to a support (e.g., the walls of a microtiter well) and then permeabilizing the cells with an appropriate permeabilizing solution. A solution containing labeled probes for K11 is then contacted with the cells and the probes allowed to hybridize. Excess probe is digested, washed away and the amount of hybridized probe measured. This approach is described in greater detail by Harris, D. W. (1996) Anal. Biochem. 243:249-256; Singer, et al. (1986) Biotechniques 4:230-250; Haase et al. (1984) Methods in Virology, vol. VII, pp. 189-226; and Nucleic Acid Hybridization: A Practical Approach (Hames, et al., eds., 1987).

A variety of so-called “real time amplification” methods or “real time quantitative PCR” methods can also be utilized to determine the quantity of K11 mRNA present in a sample. Such methods involve measuring the amount of amplification product formed during an amplification process. Fluorogenic nuclease assays are one specific example of a real time quantitation method that can be used to detect and quantitate K11 transcripts. In general such assays continuously measure PCR product accumulation using a dual-labeled fluorogenic oligonucleotide probe—an approach frequently referred to in the literature simply as the “TaqMan” method.

The probe used in such assays is typically a short (ca. 20-25 bases) polynucleotide that is labeled with two different fluorescent dyes. The 5′ terminus of the probe is typically attached to a reporter dye and the 3′ terminus is attached to a quenching dye, although the dyes can be attached at other locations on the probe as well. For measuring K11 transcript, the probe is designed to have at least substantial sequence complementarity with SEQ ID NO: 1. Upstream and downstream PCR primers that bind to regions that flank K11 gene are also added to the reaction mixture. Probes may also be made by in vitro transcription methods.

When the probe is intact, energy transfer between the two fluorophors occurs and the quencher quenches emission from the reporter. During the extension phase of PCR, the probe is cleaved by the 5′ nuclease activity of a nucleic acid polymerase such as Taq polymerase, thereby releasing the reporter dye from the polynucleotide-quencher complex and resulting in an increase of reporter emission intensity that can be measured by an appropriate detection system.

One detector which is specifically adapted for measuring fluorescence emissions such as those created during a fluorogenic assay is the ABI 7700 manufactured by Applied Biosystems, Inc. in Foster City, Calif. Computer software provided with the instrument is capable of recording the fluorescence intensity of reporter and quencher over the course of the amplification. These recorded values can then be used to calculate the increase in normalized reporter emission intensity on a continuous basis and ultimately quantify the amount of the mRNA being amplified.

Polypeptide Screening Methods

Screening for expression of the subject sequences may be based on the functional or antigenic characteristics of the protein. Protein truncation assays are useful in detecting deletions that may affect the biological activity of the protein. Various immunoassays designed to detect polymorphisms in proteins encoded by K11 may be used in screening. Where many diverse genetic mutations lead to a particular disease phenotype, functional protein assays have proven to be effective screening tools.. The activity of the encoded protein in protein assays, etc., may be determined by comparison with the wild-type protein.

Detection may utilize staining of cells or histological sections, performed in accordance with conventional methods, using antibodies or other specific binding members that specifically bind to the ischemia associated polypeptides, or ischemia pathway polypeptides. The antibodies or other specific binding members of interest are added to a cell sample, and incubated for a period of time sufficient to allow binding to the epitope, usually at least about 10 minutes. The antibody may be labeled with radioisotopes, enzymes, fluorescers, chemiluminescers, or other labels for direct detection. Alternatively, a second stage antibody or reagent is used to amplify the signal. Such reagents are well known in the art. For example, the primary antibody may be conjugated to biotin, with horseradish peroxidase-conjugated avidin added as a second stage reagent. Final detection uses a substrate that undergoes a color change in the presence of the peroxidase. The absence or presence of antibody binding may be determined by various methods, including flow cytometry of dissociated cells, microscopy, radiography, scintillation counting, etc.

An alternative method for diagnosis depends on the in vitro detection of binding between antibodies and K11 polypeptide in a lysate. Measuring the concentration of the target protein in a sample or fraction thereof may be accomplished by a variety of specific assays. A conventional sandwich type assay may be used. For example, a sandwich assay may first attach specific antibodies to an insoluble surface or support. The particular manner of binding is not crucial so long as it is compatible with the reagents and overall methods of the invention. They may be bound to the plates covalently or non-covalently, preferably non-covalently.

The insoluble supports may be any compositions to which polypeptides can be bound, which is readily separated from soluble material, and which is otherwise compatible with the overall method. The surface of such supports may be solid or porous and of any convenient shape. Examples of suitable insoluble supports to which the receptor is bound include beads, e.g. magnetic beads, membranes and microtiter plates. These are typically made of glass, plastic (e.g. polystyrene), polysaccharides, nylon or nitrocellulose. Microtiter plates are especially convenient because a large number of assays can be carried out simultaneously, using small amounts of reagents and samples.

Patient sample lysates are then added to separately assayable supports (for example, separate wells of a microtiter plate) containing antibodies. Preferably, a series of standards, containing known concentrations of the test protein is assayed in parallel with the samples or aliquots thereof to serve as controls. Preferably, each sample and standard will be added to multiple wells so that mean values can be obtained for each. The incubation time should be sufficient for binding, generally, from about 0.1 to 3 hr is sufficient. After incubation, the insoluble support is generally washed of non-bound components. Generally, a dilute non-ionic detergent medium at an appropriate pH, generally 7-8, is used as a wash medium. From one to six washes may be employed, with sufficient volume to thoroughly wash non-specifically bound proteins present in the sample.

After washing, a solution containing a second antibody is applied. The antibody will bind to one of the proteins of interest with sufficient specificity such that it can be distinguished from other components present. The second antibodies may be labeled to facilitate direct, or indirect quantification of binding. Examples of labels that permit direct measurement of second receptor binding include radiolabels, such as ³H or ¹²⁵I, fluorescers, dyes, beads, chemilumninescers, colloidal particles, and the like. Examples of labels that permit indirect measurement of binding include enzymes where the substrate may provide for a colored or fluorescent product. In a preferred embodiment, the antibodies are labeled with a covalently bound enzyme capable of providing a detectable product signal after addition of suitable substrate. Examples of suitable enzymes for use in conjugates include horseradish peroxidase, alkaline phosphatase, malate dehydrogenase and the like. Where not commercially available, such antibody-enzyme conjugates are readily produced by techniques known to those skilled in the art. The incubation time should be sufficient for the labeled ligand to bind available molecules. Generally, from about 0.1 to 3 hr is sufficient, usually 1 hr sufficing.

After the second binding step, the insoluble support is again washed free of non-specifically bound material, leaving the specific complex formed between the target protein and the specific binding member. The signal produced by the bound conjugate is detected by conventional means. Where an enzyme conjugate is used, an appropriate enzyme substrate is provided so a detectable product is formed.

Other immunoassays are known in the art and may find use as diagnostics. Ouchterlony plates provide a simple determination of antibody binding. Western blots may be performed on protein gels or protein spots on filters, using a detection system specific for the ischemia associated polypeptide, or ischemia pathway polypeptide as desired, conveniently using a labeling method as described for the sandwich assay.

In some cases, a competitive assay will be used. In addition to the patient sample, a competitor to the targeted protein is added to the reaction mix. The competitor and the ischemia associated polypeptide, or ischemia pathway polypeptide compete for binding to the specific binding partner. Usually, the competitor molecule will be labeled and detected as previously described, where the amount of competitor binding will be proportional to the amount of target protein present. The concentration of competitor molecule will be from about 10 times the maximum anticipated protein concentration to about equal concentration in order to make the most sensitive and linear range of detection.

In some embodiments, the methods are adapted for use in vivo, e.g., to locate or identify sites where ischemic cells are present. In these embodiments, a detectably-labeled moiety, e.g., an antibody, which is specific for the ischemia associated polypeptide, or ischemia pathway polypeptide is administered to an individual (e.g., by injection), and labeled cells are located using standard imaging techniques, including, but not limited to, magnetic resonance imaging, computed tomography scanning, and the like.

Activities of K11 can also be determined to detect an increase in expression. Certain assays involve detecting an increase in mitochondrial respiration in a sample from a patient potentially suffering from stroke or at risk for stroke relative to a baseline value. Assays can be conducted using isolated cells or tissue samples, or isolated mitochondrial preparations. Instead of measuring mitochondrial respiration, one can instead measure the extent of mitochondrial swelling. Methods for conducting such mitochondrial assays are known in the art and described, for example, by Salvioli et al. (1997) FEBS Lett 411:77-82; and Smiley et al. (1991) Proc. Natl. Acad. Sci. USA 88:3671-3675). Methods for conducting such assays with certain uncoupling proteins is discussed, for example, in PCT publications WO 00/17353 and WO 98/45313.

The detection methods can be provided as part of a kit. Thus, the invention further provides kits for detecting the presence of K11 mRNA, and/or a polypeptide encoded thereby, in a biological sample. Procedures using these kits can be performed by clinical laboratories, experimental laboratories, medical practitioners, or private individuals. The kits of the invention for detecting a polypeptide comprise a moiety that specifically binds the polypeptide, which may be a specific antibody. The kits of the invention for detecting a nucleic acid comprise a moiety that specifically hybridizes to such a nucleic acid. The kit may optionally provide additional components that are useful in the procedure, including, but not limited to, buffers, developing reagents, labels, reacting surfaces, means for detection, control samples, standards, instructions, and interpretive information.

Time Course Analyses

Certain prognostic methods of assessing a patient's risk of stroke involve monitoring expression levels for a patient susceptible to stroke, to track whether there is an increase in expression of an K11 over time. An increase in expression over time can indicate that the individual is at increased risk for stroke. As with other measures, the expression level for the patient at risk for stroke is compared against a baseline value. The baseline in such analyses can be a prior value determined for the same individual or a statistical value (e.g., mean or average) determined for a control group (e.g., a population of individuals with no apparent neurological risk factors). An individual showing a statistically significant increase in ischemia associated gene expression levels over time can prompt the individual's physician to take prophylactic measures to lessen the individual's potential for stroke. For example, the physician can recommend certain life style changes (e.g., improved diet, exercise program) to reduce the risk of stroke. Alternatively, or in addition, the physician can prescribe medicines to reduce the stroke risk.

Antibody Conjugates

The anti-K11 antibodies for use in the present invention may have utility without conjugation. Such antibodies, which may be selected as described above, may be utilized without conjugation as a therapeutic agent to inhibit K11 activity, and for diagnostic purposes. In another embodiment of the invention, K11 specific antibodies, which may or may not alter the activity of the target polypeptide, are conjugated to imaging agents, which add functionality to the antibody. “Imaging moiety” (I) is a moiety that can be utilized to increase contrast between a tumor and the surrounding healthy tissue in a visualization technique (e.g., radiography, positron-emission tomography, magnetic resonance imaging, direct or indirect visual inspection). Thus, suitable imaging moieties include radiography moieties (e.g. heavy metals and radiation emitting moieties), positron emitting moieties, magnetic resonance contrast moieties, and optically visible moieties (e.g., fluorescent or visible-spectrum dyes, visible particles, etc.). It will be appreciated by one of ordinary skill that some overlap exists between therapeutic and imaging moieties. For instance ²¹²Pb and ²¹²Bi are both useful radioisotopes for therapeutic compositions, but are also electron-dense, and thus provide contrast for X-ray radiographic imaging techniques, and can also be utilized in scintillation imaging techniques.

In general, therapeutic or imaging agents may be conjugated to the anti-K11 moiety by any suitable technique, with appropriate consideration of the need for pharmokinetic stability and reduced overall toxicity to the patient. A therapeutic agent may be coupled to a suitable antibody moiety either directly or indirectly (e.g. via a linker group). A direct reaction between an agent and an antibody is possible when each possesses a functional group capable of reacting with the other. For example, a nucleophilic group, such as an amino or sulfhydryl group, may be capable of reacting with a carbonyl-containing group, such as an anhydride or an acid halide, or with an alkyl group containing a good leaving group (e.g., a halide). Alternatively, a suitable chemical linker group may be used. A linker group can function as a spacer to distance an antibody from an agent in order to avoid interference with binding capabilities. A linker group can also serve to increase the chemical reactivity of a substituent on a moiety or an antibody, and thus increase the coupling efficiency. An increase in chemical reactivity may also facilitate the use of moieties, or functional groups on moieties, which otherwise would not be possible.

Suitable linkage chemistries include maleimidyl linkers and alkyl halide linkers (which react with a sulfhydryl on the antibody moiety) and succinimidyl linkers (which react with a primary amine on the antibody moiety). Several primary amine and sulfhydryl groups are present on immunoglobulins, and additional groups may be designed into recombinant immunoglobulin molecules. It will be evident to those skilled in the art that a variety of bifunctional or polyfunctional reagents, both homo- and hetero-functional (such as those described in the catalog of the Pierce Chemical Co., Rockford, Ill.), may be employed as a linker group. Coupling may be effected, for example, through amino groups, carboxyl groups, sulfhydryl groups or oxidized carbohydrate residues. There are numerous references describing such methodology, e.g., U.S. Pat. No. 4,671,958. As an alternative coupling method, cytotoxic or imaging moieties may be coupled to the anti-TBT antibody moiety through a an oxidized carbohydrate group at a glycosylation site, as described in U.S. Pat. Nos. 5,057,313 and 5,156,840. Yet another alternative method of coupling the antibody moiety to the cytotoxic or imaging moiety is by the use of a non-covalent binding pair, such as streptavidin/biotin, or avidin/biotin. In these embodiments, one member of the pair is covalently coupled to the antibody moiety and the other member of the binding pair is covalently coupled to the cytotoxic or imaging moiety.

Two or more moieties may be conjugated to an antibody, where the, conjugated moieties are the same or different. By poly-derivatizing the anti-K11 antibody, several strategies can be simultaneously implemented; an antibody may be made useful as a contrasting agent for several visualization techniques; or a therapeutic antibody may be labeled for tracking by a visualization technique. Immunoconjugates with more than one moiety may be prepared in a variety of ways. For example, more than one moiety may be coupled directly to an antibody molecule, or linkers which provide multiple sites for attachment (e.g., dendrimers) can be used. Alternatively, a carrier with the capacity to hold more than one cytotoxic or imaging moiety can be used.

A carrier may bear the moiety in a variety of ways, including covalent bonding either directly or via a linker group, and non-covalent associations. Suitable covalent-bond carriers include proteins such as albumins (e.g., U.S. Pat. No. 4,507,234), peptides, and polysaccharides such as aminodextran (e.g., U.S. Pat. No. 4,699,784), each of which have multiple sites for the attachment of moieties. A carrier may also bear an agent by non-covalent associations, such as non-covalent bonding or by encapsulation, such as within a liposome vesicle (e.g., U.S. Pat. Nos. 4,429,008 and 4,873,088). Encapsulation carriers are especially useful for imaging moiety conjugation to anti-K11antibody moieties for use in the invention, as a sufficient amount of the imaging moiety (dye, magnetic resonance contrast reagent, etc.) for detection may be more easily associated with the antibody moiety. In addition, encapsulation carriers are also useful in chemotoxic therapeutic embodiments, as they can allow the therapeutic compositions to gradually release a chemotoxic moiety over time while concentrating it in the vicinity of the tumor cells.

Carriers and linkers specific for radionuclide agents include radiohalogenated small molecules and chelating compounds. For example, U.S. Pat. No. 4,735,792 discloses representative radiohalogenated small molecules and their synthesis. A radionuclide chelate may be formed from chelating compounds that include those containing nitrogen and sulfur atoms as the donor atoms for binding the metal, or metal oxide, radionuclide. For example, U.S. Pat. No. 4,673,562, to Davison et al. discloses representative chelating compounds and their synthesis. Such chelation carriers are also useful for magnetic spin contrast ions for use in magnetic resonance imaging tumor visualization methods, and for the chelation of heavy metal ions for use in radiographic visualization methods.

Preferred radiographic moieties for use as imaging moieties in the present invention include compounds and chelates with relatively large atoms, such as gold, iridium, technetium, barium, thallium, iodine, and their isotopes. It is preferred that less toxic radiographic imaging moieties, such as iodine or iodine isotopes, be utilized in the compositions and methods of the invention. Examples of compositions that may be utilized for x-ray radiography are described in U.S. Pat. No. 5,709,846, incorporated fully herein by reference. Such moieties may be conjugated to the anti-K11 antibody moiety through an acceptable chemical linker or chelation carrier. In addition, radionuclides that emit radiation capable of penetrating the skull may be useful for scintillation imaging techniques. Suitable radionuclides for conjugation include ⁹⁹Tc, ¹¹¹In, and ⁶⁷Ga. Positron emitting moieties for use in the present invention include ¹⁸F, which can be easily conjugated by a fluorination reaction with the anti-K11antibody moiety according to the method described in U.S. Pat. No. 6,187,284.

Preferred magnetic resonance contrast moieties include chelates of chromium(III), manganese(II), iron(II), nickel(II), copper(II), praseodymium(III), neodymium(III), samarium(III) and ytterbium(III) ion. Because of their very strong magnetic moment, the gadolinium(III), terbium(III), dysprosium(III), holmium(III), erbium(III), and iron(III) ions are especially preferred. Examples of such chelates, suitable for magnetic resonance spin imaging, are described in U.S. Pat. No. 5,733,522, incorporated fully herein by reference. Nuclear spin contrast chelates may be conjugated to the anti-K11 antibody moieties through a suitable chemical linker.

Optically visible moieties for use as imaging moieties include fluorescent dyes, or visible-spectrum dyes, visible particles, and other visible labeling moieties. Fluorescent dyes such as ALEXA dyes, fluorescein, coumarin, rhodamine, bodipy Texas red, and cyanine dyes, are useful when sufficient excitation energy can be provided to the site to be inspected visually. Endoscopic visualization procedures may be more compatible with the use of such labels. For many procedures where imaging agents are useful, such as during an operation to resect a brain tumor, visible spectrum dyes are preferred. Acceptable dyes include FDA-approved food dyes and colors, which are non-toxic, although pharmaceutically acceptable dyes which have been approved for internal administration are preferred. In preferred embodiments, such dyes are encapsulated in carrier moieties, which are in turn conjugated to the anti-K11 antibody. Alternatively, visible particles, such as colloidal gold particles or latex particles, may be coupled to the anti-K11 antibody moiety via a suitable chemical linker.

Therapeutic/Prophylactic Treatment Methods

Agents that modulate activity of K11 provide a point of therapeutic or prophylactic intervention. Numerous agents are useful in modulating this activity, including agents that directly modulate expression, e.g. expression vectors, antisense specific for the targeted protein; and agents that act on the protein, e.g. specific antibodies and analogs thereof, small organic molecules that block catalytic activity, etc.

The genes, gene fragments, or the encoded protein or protein fragments are useful in therapy to treat disorders associated with defects in sequence or expression. From a therapeutic point of view, modulating activity has a therapeutic effect on a number of neurodegenerative disorders. Antisense sequences may be administered to inhibit expression. Pseudo-substrate inhibitors, for example, a peptide that mimics a substrate for the protein may be used to inhibit activity. Other inhibitors are identified by screening for biological activity in a functional assay, e.g. in vitro or in vivo protein activity. Alternatively, expression can be upregulated by introduction of an expression vector, enhancing expression, providing molecules that mimic the activity of the targeted polypeptide, etc.

Expression vectors may be used to introduce the target gene into a cell. Such vectors generally have convenient restriction sites located near the promoter sequence to provide for the insertion of nucleic acid sequences. Transcription cassettes may be prepared comprising a transcription initiation region, the target gene or fragment thereof, and a transcriptional termination region. The transcription cassettes may be introduced into a variety of vectors, e.g. plasmid; retrovirus, e.g. lentivirus; adenovirus; and the like, where the vectors are able to transiently or stably be maintained in the cells, usually for a period of at least about one day, more usually for a period of at least about several days to several weeks.

The gene or protein may be introduced into tissues or host cells by any number of routes, including viral infection, microinjection, or fusion of vesicles. Jet injection may also be used for intramuscular administration, as described by Furth et al. (1992) Anal Biochem 205:365-368. The DNA may be coated onto gold microparticles, and delivered intradermally by a particle bombardment device, or “gene gun” as described in the literature (see, for example, Tang et al. (1992) Nature 356:152-154), where gold micro projectiles are coated with the protein or DNA, then bombarded into skin cells.

Methods can be designed to selectively deliver nucleic acids to certain cells. Examples of such cells include, neurons, microglia, astrocytes, endothelial cells, oligodendrocytes, etc. Certain treatment methods are designed to selectively express an expression vector to neuron cells and/or target the nucleic acid for delivery to nerve cells (microglia, astrocytes, endothelial cells, oligodendrocytes). One technique for achieving selective expression in nerve cells is to operably link the coding sequence to a promoter that is primarily active in nerve cells. Examples of such promoters include, but are not limited to, prion protein promoter, calcium-calmodulin dependent protein kinase promoter. Alternatively, or in addition, the nucleic acid can be administered with an agent that targets the nucleic acid to nerve cells. For instance, the nucleic acid can be administered with an antibody that specifically binds to a cell-surface antigen on the nerve cells or a ligand for a receptor on neuronal cells.

When liposomes are utilized, substrates that bind to a cell-surface membrane protein associated with endocytosis can be attached to the liposome to target the liposome to nerve cells and to facilitate uptake. Examples of proteins that can be attached include capsid proteins or fragments thereof that bind to nerve cells, antibodies that specifically bind to cell-surface proteins on nerve cells that undergo internalization in cycling and proteins that target intracellular localizations within nerve cells (see, e.g., Wu et al. (1987) J. Biol. Chem. 262:4429-4432; and Wagner, et al. (1990) Proc. Natl. Acad. Sci. USA 87:3410-3414). Gene marking and gene therapy protocols are reviewed by Anderson et al. (1992) Science 256:808-813.

Various other delivery options can also be utilized. For instance, a nucleic acid containing K11 gene can be injected directly into the cerebrospinal fluid. Alternatively, such nucleic acids can be administered by intraventricular injections.

Antisense molecules can be used to down-regulate expression in cells. The antisense reagent may be antisense oligonucleotides (ODN), particularly synthetic ODN having chemical modifications from native nucleic acids, or nucleic acid constructs that express such antisense molecules as RNA. The antisense sequence is complementary to the mRNA of the targeted gene, and inhibits expression of the targeted gene products. Antisense molecules inhibit gene expression through various mechanisms, e.g. by reducing the amount of mRNA available for translation, through activation of RNAse H, or steric hindrance. One or a combination of antisense molecules may be administered, where a combination may comprise multiple different sequences.

Antisense molecules may be produced by expression of all or a part of the target gene sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, usually at least about 12, more usually at least about 20 nucleotides in length, and not more than about 500, usually not more than about 50, more usually not more than about 35 nucleotides in length, where the length is governed by efficiency of inhibition, specificity, including absence of cross-reactivity, and the like. It has been found that short oligonucleotides, of from 7 to 8 bases in length, can be strong and selective inhibitors of gene expression (see Wagner et al. (1996) Nature Biotechnology 14:840-844).

A specific region or regions of the endogenous sense strand mRNA sequence is chosen to be complemented by the antisense sequence. Selection of a specific sequence for the oligonucleotide may use an empirical method, where several candidate sequences are assayed for inhibition of expression of the target gene in vitro or in an animal model. A combination of sequences may also be used, where several regions of the mRNA sequence are selected for antisense complementation.

Antisense oligonucleotides may be chemically synthesized by methods known in the art (see Wagner et al. (1993) supra. and Milligan et al., supra.) Preferred oligonucleotides are chemically modified from the native phosphodiester structure, in order to increase their intracellular stability and binding affinity. A number of such modifications have been described in the literature, which alter the chemistry of the backbone, sugars or heterocyclic bases.

Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH2-5′-O-phosphonate and 3′-NH-5′-O-phosphoroamidate. Peptide nucleic acids replace the entire ribose phosphodiester backbone with a peptide linkage. Sugar modifications are also used to enhance stability and affinity. The alpha.-anomer of deoxyribose may be used, where the base is inverted with respect to the natural .beta.-anomer. The 2′-OH of the ribose sugar may be altered to form 2′-O-methyl or 2′-O-allyl sugars, which provides resistance to degradation without comprising affinity. Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

Compound Screening

Compound screening may be performed using an in vitro model, a genetically altered cell or animal, or purified K11 protein. One can identify ligands or substrates that bind to, modulate or mimic the action of the encoded polypeptide. Compounds are also identified that affect the cleavage of K11.

The polypeptides include those encoded by K11, as well as by nucleic acids that, by virtue of the degeneracy of the genetic code, are not identical in sequence to the disclosed nucleic acids, and variants thereof. Variant polypeptides can include amino acid (aa) substitutions, additions or deletions. The amino acid substitutions can be conservative amino acid substitutions or substitutions to eliminate non-essential amino acids, such as to alter a glycosylation site, a phosphorylation site or an acetylation site, or to minimize misfolding by substitution or deletion of one or more cysteine residues that are not necessary for function. Variants can be designed so as to retain or have enhanced biological activity of a particular region of the protein (e.g., a functional domain and/or, where the polypeptide is a member of a protein family, a region associated with a consensus sequence). Variants also include fragments of the polypeptides disclosed herein, particularly biologically active fragments and/or fragments corresponding to functional domains. Fragments of interest will typically be at least about 10 aa to at least about 15 aa in length, usually at least about 50 aa in length, and can be as long as 300 aa in length or longer, but will usually not exceed about 550 aa in length, where the fragment will have a contiguous stretch of amino acids that is identical to a polypeptide encoded by K11, or a homolog thereof.

Transgenic animals or cells derived therefrom are also used in compound screening. Transgenic animals may be made through homologous recombination, where the normal locus corresponding to K11 is altered. Alternatively, a nucleic acid construct is randomly integrated into the genome. Vectors for stable integration include plasmids, retroviruses and other animal viruses, YACs, and the like. A series of small deletions and/or substitutions may be made in the coding sequence to determine the role of different exons in protein activity, oncogenesis, signal transduction, etc. Of interest is the use of K11 to construct transgenic animal models for ischemia, where expression of the corresponding protein is specifically reduced or absent. Specific constructs of interest include antisense sequences that block expression of the targeted gene and expression of dominant negative mutations. A detectable marker, such as lac Z may be introduced into the locus of interest, where up-regulation of expression will result in an easily detected change in phenotype. One may also provide for expression of the target gene or variants thereof in cells or tissues where it is not normally expressed or at abnormal times of development. By providing expression of the target protein in cells in which it is not normally produced, one can induce changes in cell behavior.

Compound screening identifies agents that modulate function of K11. Of particular interest are screening assays for agents that have a low toxicity for human cells. A wide variety of assays may be used for this purpose, including labeled in vitro protein-protein binding assays, electrophoretic mobility shift assays, immunoassays for protein binding, and the like. Knowledge of the 3-dimensional structure of the encoded protein, derived from crystallization of purified recombinant protein, could lead to the rational design of small drugs that specifically inhibit activity. These drugs may be directed at specific domains.

The term “agent” as used herein describes any molecule, e.g. protein or pharmaceutical, with the capability of altering or mimicking the physiological function K11. Generally a plurality of assay mixtures are run in parallel with different agent concentrations to obtain a differential response to the various concentrations. Typically one of these concentrations serves as a negative control, i.e. at zero concentration or below the level of detection.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Test agents can be obtained from libraries, such as natural product libraries or combinatorial libraries, for example. A number of different types of combinatorial libraries and methods for preparing such libraries have been described, including for example, PCT publications WO 93/06121, WO 95/12608, WO 95/35503, WO 94/08051 and WO 95/30642, each of which is incorporated herein by reference.

Where the screening assay is a binding assay, one or more of the molecules may be joined to a label, where the label can directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin, etc. For the specific binding members, the complementary member would normally be labeled with a molecule that provides for detection, in accordance with known procedures.

A variety of other reagents may be included in the screening assay. These include reagents like salts, neutral proteins, e.g. albumin, detergents, etc that are used to facilitate optimal protein-protein binding and/or reduce non-specific or background interactions. Reagents that improve the efficiency of the assay, such as protease inhibitors, nuclease inhibitors, anti-microbial agents, etc. may be used. The mixture of components are added in any order that provides for the requisite binding. Incubations are performed at any suitable temperature, typically between 4 and 40° C. Incubation periods are selected for optimum activity, but may also be optimized to facilitate rapid high-throughput screening. Typically between 0.1 and 1 hours will be sufficient.

Preliminary screens can be conducted by screening for compounds capable of binding to K11, as at least some of the compounds so identified are likely K11 inhibitors. The binding assays usually involve contacting K11 protein with one or more test compounds and allowing sufficient time for the protein and test compounds to form a binding complex. Any binding complexes formed can be detected using any of a number of established analytical techniques. Protein binding assays include, but are not limited to, methods that measure co-precipitation, co-migration on non-denaturing SDS-polyacrylamide gels, and co-migration on Western blots (see, e.g., Bennet, J. P. and Yamamura, H. I. (1985) “Neurotransmitter, Hormone or Drug Receptor Binding Methods,” in Neurotransmitter Receptor Binding (Yamamura, H. I., et al., eds.), pp. 61-89. The K11 protein utilized in such assays can be naturally expressed, cloned or synthesized.

Certain screening methods involve screening for a compound that modulates the expression of K11 gene. Such methods generally involve conducting cell-based assays in which test compounds are contacted with one or more cells expressing K11 and then detecting an increase in K11 gene expression (either transcript or translation product). Some assays are performed with neuron cells that express endogenous K11 (e.g., cortical neuron cells, glial cells or microglial cells). Other expression assays are conducted with non-neuronal cells that express an exogenous K11 sequence.

K11 expression can be detected in a number of different ways. The expression level of a K11 in a cell can be determined by probing the mRNA expressed in a cell with a probe that specifically hybridizes with a transcript (or complementary nucleic acid derived therefrom) of K11. Probing can be conducted by lysing the cells and conducting Northern blots or without lysing the cells using in situ-hybridization techniques (see above). Alternatively, K11 can be detected using immunological methods in which a cell lysate is probe with antibodies that specifically bind to K11 protein.

Other cell-based assays are reporter assays conducted with cells that do not express K11. Certain of these assays are conducted with a heterologous nucleic acid construct that includes a K11 promoter that is operably linked to a reporter gene that encodes a detectable product. A number of different reporter genes can be utilized. Some reporters are inherently detectable. An example of such a reporter is green fluorescent protein that emits fluorescence that can be detected with a fluorescence detector. Other reporters generate a detectable product. Often such reporters are enzymes. Exemplary enzyme reporters include, but are not limited to, β-glucuronidase, CAT (chloramphenicol acetyl transferase; Alton and Vapnek (1979) Nature 282:864-869), luciferase, β-galactosidase and alkaline phosphatase (Toh, et al. (1980) Eur. J. Biochem. 182:231-238; and Hall et al. (1983) J. Mol. Appl. Gen. 2:101).

In these assays, cells harboring the reporter construct are contacted with a test compound. A test compound that either activates the promoter by binding to it or triggers a cascade that produces a molecule that activates the promoter causes expression of the detectable reporter. Certain other reporter assays are conducted with cells that harbor a heterologous construct that includes a transcriptional control element that activates expression of K11 and a reporter operably linked thereto. Here, too, an agent that binds to the transcriptional control element to activate expression of the reporter or that triggers the formation of an agent that binds to the transcriptional control element to activate reporter expression, can be identified by the generation of signal associated with reporter expression.

The level of expression or activity can be compared to a baseline value. As indicated above, the baseline value can be a value for a control sample or a statistical value that is representative of K11 expression levels for a control population (e.g., healthy individuals not at risk for neurological injury such as stroke). Expression levels can also be determined for cells that do not express a K11 as a negative control. Such cells generally are otherwise substantially genetically the same as the test cells.

A variety of different types of cells can be utilized in the reporter assays. Certain cells are nerve cells that express endogenous K11. Cells not expressing such K11 can be prokaryotic, but preferably are eukaryotic. The eukaryotic cells can be any of the cells typically utilized in generating cells that harbor recombinant nucleic acid constructs. Exemplary eukaryotic cells include, but are not limited to, yeast, and various higher eukaryotic cells such as the COS, CHO and HeLa cell lines.

Various controls can be conducted to ensure that an observed activity is authentic including running parallel reactions with cells that lack the reporter construct or by not contacting a cell harboring the reporter construct with test compound. Compounds can also be further validated as described below.

Compounds that are initially identified by any of the foregoing screening methods can be further tested to validate the apparent activity. The basic format of such methods involves administering a lead compound identified during an initial screen to an animal that serves as a model for humans and then determining if K11 is in fact upregulated. The animal models utilized in validation studies generally are mammals. Specific examples of suitable animals include, but are not limited to, primates, mice, and rats.

Certain methods are designed to test not only the ability of a lead compound to modulate K11 activity in an animal model, but to provide protection after the animal has undergone transient ischemia for a longer period of time than shown to provide a protective effect. In such methods, a lead compound is administered to the model animal (i.e., an animal, typically a mammal, other than a human). The animal is subsequently subjected to transient ischemia for a period longer in duration than that shown to provide a protective effect. The conditions causing the ischemia are halted and the K11 activity monitored to identify those compounds still able to modulate K11 activity above a baseline level. Compounds able to modulate K11 gene expression beyond the time period in which the K11 is upregulated in preconditioning models are good candidates for further study.

Active test agents identified by the screening methods described herein that modulate K11 activity can serve as lead compounds for the synthesis of analog compounds. Typically, the analog compounds are synthesized to have an electronic configuration and a molecular conformation similar to that of the lead compound. Identification of analog compounds can be performed through use of techniques such as self-consistent field (SCF) analysis, configuration interaction (CI) analysis, and normal mode dynamics analysis. Computer programs for implementing these techniques are available. See, e.g., Rein et al., (1989) Computer-Assisted Modeling of Receptor-Ligand Interactions (Alan Liss, N.Y.).

Once analogs have been prepared, they can be screened using the methods disclosed herein to identify those analogs that exhibit an increased ability to modulate K11 activity. Such compounds can then be subjected to further analysis to identify those compounds that have the greatest potential as pharmaceutical agents. Alternatively, analogs shown to have activity through the screening methods can serve as lead compounds in the preparation of still further analogs, which can be screened by the methods described herein. The cycle of screening, synthesizing analogs and re-screening can be repeated multiple times.

Pharmaceutical Compositions

Compounds identified by the screening methods described above and analogs thereof can serve as the active ingredient in pharmaceutical compositions formulated for the treatment of various neurological disorders, including stroke. The compositions can also include various other agents to enhance delivery and efficacy. For instance, compositions can include agents capable of increasing the permeability of the blood/brain barrier. The compositions can also include various agents to enhance delivery and stability of the active ingredients.

Thus, for example, the compositions can also include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from cell culture and/or animal studies can be used in formulating a range of dosages for humans. The dosage of the active ingredient typically lines within a range of circulating concentrations that include the ED₅₀ with little or no toxicity. The dosage can vary within this range depending upon the dosage form employed and the route of administration utilized.

The pharmaceutical compositions described herein can be administered in a variety of different ways. Examples include administering a composition containing a pharmaceutically acceptable carrier via oral, intranasal, rectal, topical, intraperitoneal, intravenous, intramuscular, subcutaneous, subdermal, transdermal, intrathecal, and intracranial methods.

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

The active ingredient, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen.

Suitable formulations for rectal administration include, for example, suppositories, which consist of the packaged active ingredient with a suppository base. Suitable suppository bases include natural or synthetic triglycerides or paraffin hydrocarbons. In addition, it is also possible to use gelatin rectal capsules which consist of a combination of the packaged active ingredient with a base, including, for example, liquid triglycerides, polyethylene glycols, and paraffin hydrocarbons.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

Experimental

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### EXAMPLE 1

Materials and Methods

Animal Preparation and experimental Groups. The procedures for transient MCAO were performed as described previously (Zhao et al. (1997) J Cereb Blood Flow Metab. 17(12):1281-90) and are summarized briefly below. All experimental procedures were approved by the Malmö/Lund Ethical Committee on Animal Experiments at Lund University under the supervision of the Swedish Department of Agriculture. Male Wistar rats (Möllegaards Breeding Center, Copenhagen), weighing 310-350 g, were fasted overnight but had free access to water. Anesthesia was induced by inhalation of 3% halothane in N₂O:O₂ (70%:30%), whereafter the animals were intubated. They were then ventilated on 1.0-1.5% halothane in N₂O:O₂ during operation. The tail artery was cannulated for blood sampling and blood pressure monitoring. Blood pressure, PaO₂, PaCO₂, pH, and blood glucose were measured, and 0.1 ml of heparin (300 units×ml⁻¹) was given through the tail artery just before induction of ischemia. A surgical mid-line incision was made to expose the right common, internal, and external carotid arteries. The external carotid artery was ligated. The common carotid artery was closed by a ligature, and the internal carotid artery was temporarily closed by a microvascular clip. A small incision was made in the common carotid artery, and a nylon filament, which had a distal cylinder of silicon rubber (diameter 0.28 mm), was inserted into the internal carotid artery through the common carotid artery. The filament was further advanced 19 mm to occlude the origin of the middle cerebral artery (MCA). When the middle cerebral artery occlusion (MCAO) had been performed, animals were extubated and allowed to wake up and resume spontaneous breathing. In the group aimed for recirculation, the animals were reanesthetized with halothane after 2hrs of MCAO, and the filament was withdrawn. During the operation, an electrical temperature probe was inserted 7 cm into the rectum to monitor core temperature, which was regularly maintained at 37° C. After the operation, the animals were cooled by an air cooling system to avoid the hypothermia which would otherwise occur and to keep core temperature close to normal levels during and following MCAO. All animals were tested for neurological status according to the neurological examination grading system described by Bederson et al. (1986) Stroke 17(3):472-6. Only rats that consistently circled toward the paretic side (grade 3) during 2 h of MCAO (1 hr of MCAO in 1 hr group) were included in this study.

Animals sacrificed after 2hrs of MCAO and 0′, 45′, 1.5 h, 3 h, 4.5 h, 6 h, 9 h, 12 h, 15 h, 18 h, 21 h, 24 h, 48 h reperfusion by decapitation. Brain samples generated for cDNA and micro array experiments were dissected out at −20° C. from coronal sections through anatomic regions of neocortex to core, penumbral, penumbra 2 and contra-lateral side of cortex. The samples were stored at −80° C. until homogenization. Also, animals generated for in situ hybridization were sacrificed in the same way and the brain were taken out and frozen in imbedding media at −50° C. and stored at −80° C. before sectioning.

The ischemic preconditioning (3 min of ischemia) and the second subsequent ischemia with or without preconditioning were done acording to Shamloo et al. (1999). Breifely male Wistar rats, weighing 300-340 g were used. The animals were fasted overnight with access to water before surgery. We employed the two-vessel occlusion model of global cerebral ischemia in our experiments (Smith et al. 1984). Anesthesia was induced with 3% halothane in N₂O/O₂ (70:30). The animals were intubated and connected to a respirator, and the halothane concentration reduced to 1.2-1.5%. A tail artery and a tail vein were cannulated for blood pressure recordings, blood sampling, and drug infusions. In order to allow rapid withdrawal of blood for induction of ischemia, a soft silastic catheter was inserted into the inferior caval vein through the right jugular vein. The common carotid arteries were isolated and encircled with loose ligatures. Electrodes for recording of bipolar EEG were inserted into the temporal muscles, and EEG activity was recorded prior to, during and following ischemia. Following surgery, the halothane was decreased to 0.3-0.5% and the ventilation and O₂ supply was adjusted to give an arterial PCO₂ of 35-40 mm Hg and a PO₂ of close to 100 mm Hg. Prior to the first blood gas measurement, 90 IU·kg⁻¹ heparin was given. Norcuron (2mg·h⁻¹) was administered as muscle relaxant. A heating lamp and pad were used to keep the head and body temperature at 37° C. during the ischemic episode. Ischemia was induced by withdrawal of blood through the central venous catheter to a blood pressure to 50 mmHg followed by clamping of the common carotid arteries with atraumatic clips. Ischemia was terminated by reinfusion of the blood and release of the carotid clamps. To prevent systemic acidosis induced after termination of ischemia, 0.5 ml of a 0.6 M NaHCO₃ solution was given intravenously. The Norcuron infusion was discontinued. When the animals regained spontaneous breathing, they were extubated and disconnected from the respirator. During recovery following ischemia, the rats were housed in cages with access to water and food. The second ischemic event was induced in a similar manner at 2 days following the sublethal ischemia. Sham operated animals were treated similarly as those subjected to ischemia, except for the occlusion of the carotid arteries and the induction of hypotension. The animal were terminated after 4, 12,18,24,48 hours of revoery following 3 min ischemia or 12,18 and 24 hours after the second subseqeunt ischemia as already described above for in situ hybridization.

Total RNA from these samples was prepared using Trizol (Life Technologies). mRNA was selected using Oligotex Kit (Quiagen). The poly A⁺ RNA was reverse transcribed using an Oligo dT priming method and converted into double strand cDNA (ds cDNA) using standard methods. The library was enhanced for sequences of interest using a tester/driver subtraction protocol (see U.S. patent application Ser. No. 09/627,362, filed Jul. 28, 2000; and U.S. patent application Ser. No. 09/628,202, filed Jul. 28, 2000, which are hereby incorporated by reference in their entirety into this application for all purposes.) The samples taken from MCAO were used as the tester, and the driver samples were controls, with a sham procedure.

The cDNA insert of each clone was PCR-amplified using vector-specific primers. PCR products were verified by gel electrophoresis. PCR products were spotted in sextuplicate on nylon membranes to produce the array. The cDNA fragments were denatured by wetting the membrane in a solution of 0.5M sodium hydroxide, 1.5M sodium chloride to allow better availability for hybridization, neutralized and crosslinked by ultraviolet light (Stratalinker, Stratagene).

About 5000 upregulated genes were selected after bioinformatic analysis of array data. Selection was based on expression upregulation of ≧1.8 and cv of 0.2. The selected clones were picked, amplified by PCR, and re-printed on nylon membranes for profiling arrays. Also, plasmid vectors with gene inserts were generated for the selected genes. These inserts were later used in in situ hybridization and dsRNAi experiments as template for in vitro transcription.

The profiling arrays were probed with probes from different penumbra regions and recovery times: 0′rec, 45′ rec, 1.5 h rec, 3 h rec, 4.5 h rec, 6 h rec, 9 h rec, 12 h rec, 15 h rec, 18 h rec, 21 h rec, 24 h rec and 48 h rec. Clones that were upregulated at ≧2 timepoints with a total induction of ≧1.8 and cv of 0.2 were selected for further analysis.

Results

K11 was found to be up-regulated by ischemia at multiple time points. The corresponding EST sequence matches the human sequence from chromosome 11. To study the expression of K11, the sequence was used as a template for generation of RNA probe. We observed a high expression in whole ischemic hemisphere following the ischemic insult. The K11 remains up-regulated up to 48 hours recovery after 2 hour of MCAO in the ischemic regions.

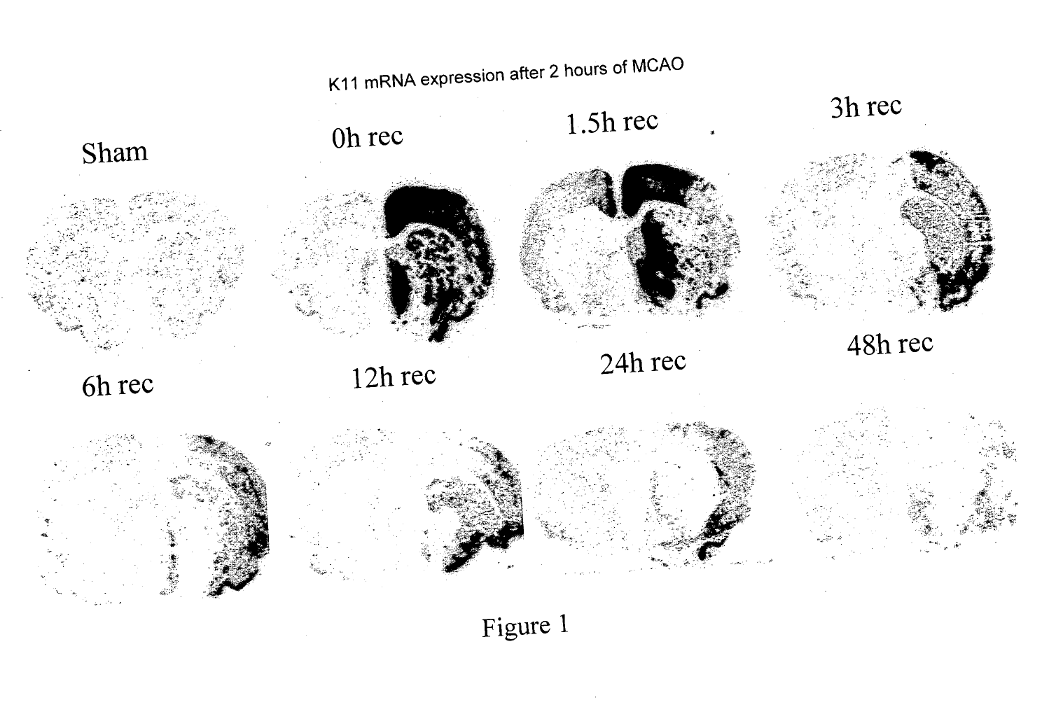

Animals were subjected to 2 hours of MCAO and 0,1.5, 3, 6, 12, 24 and 48 hours of recovery or a sham surgery and 1.5 and 24 hour of recovery. In situ hybridization analysis shows that the K11 mRNA is expressed highly in the CA1 region of hippocampus, and cortex, in un-stimulated brains. Moreover the highest expression level was observed in the cortex. When animals were subjected to 2 hours of MCAO the mRNA level of K11 was significantly and immediately increased in striatum and cortex. The over expression was obvious during entire time of reperfusion. Early after the ischemic insult the up regulation of mRNA was observed in both infarct and pre-infarct regions however after 3 hours of reperfusion the induction was just observed in the core and immediate penumbra regions of brain (see FIG. 1)

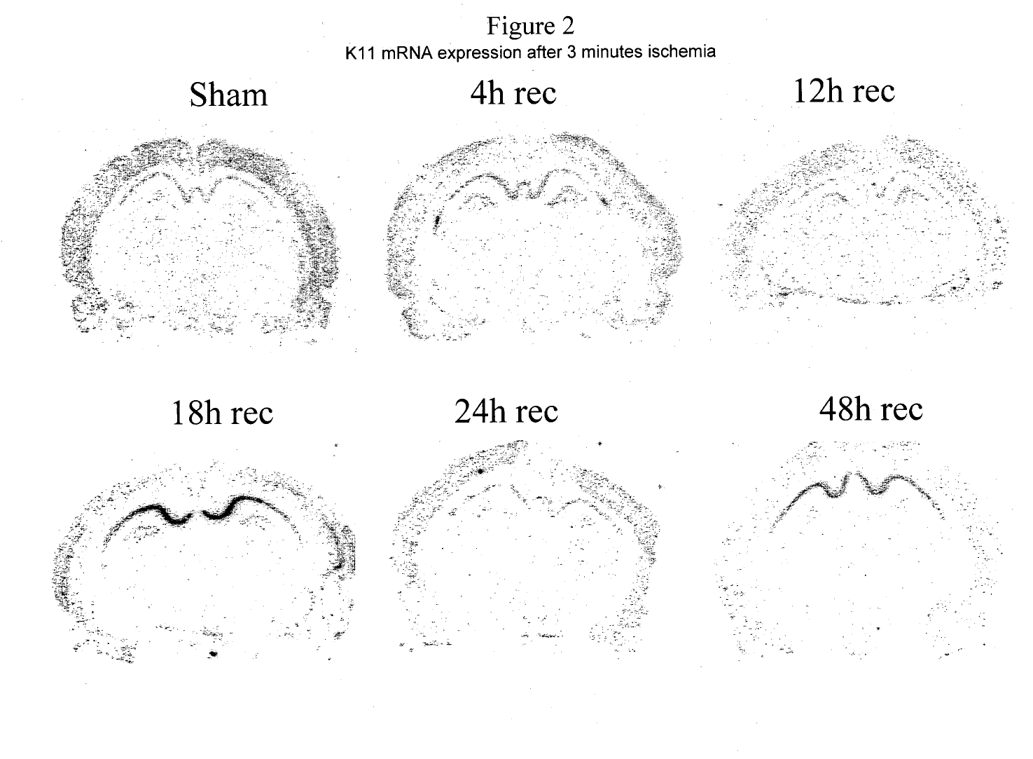

Animals were subjected to 3 min of global ischemia and 4, 12, 18, 24, and 48 hours of recovery or a sham surgery and 48 hours of recovery. Three minutes of ischemia induced a significant up regulation of K11 mRNA in CA1 region of hippocampus, at 18 and 48 hours of recovery (see FIG. 2).

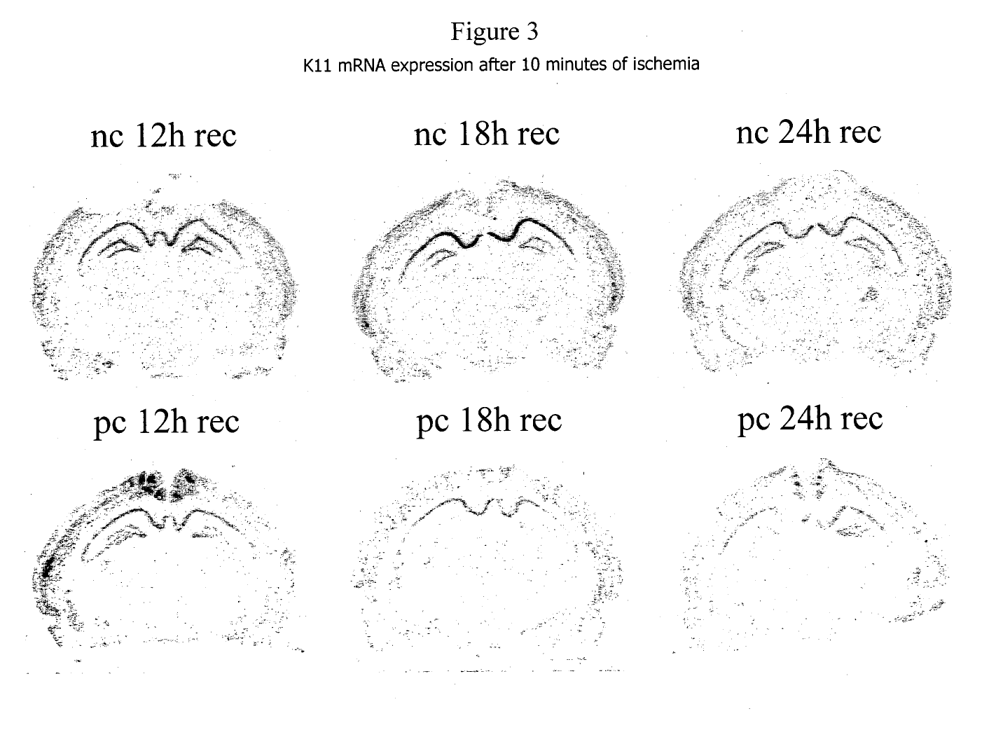

Animals were subjected to 10 min of ischemia and 12, 18 and 24 hours of recovery with (pci) or without (nci) 3 min of preconditioning. 10 min of ischemia when it is preconditioned with 3 min ischemia will not induce any neuronal damage to the brain. However, when the 10 min ischemia is not preconditioned with the 3 min of ischemia a massive neuronal damage will be observed in CA1 region of hippocampus. 10 minutes of ischemia without preconditioning induced a significant and persistent up regulation of K11 mRNA in hippocampus at 12, 18 and 24 hours of recovery. In contrast, when the brains were preconditioned with 3 minutes ischemia two days prior to the 10 minutes ischemia no significant up regulation of K11 was detected in hippocampus at any time points, see FIG. 3.

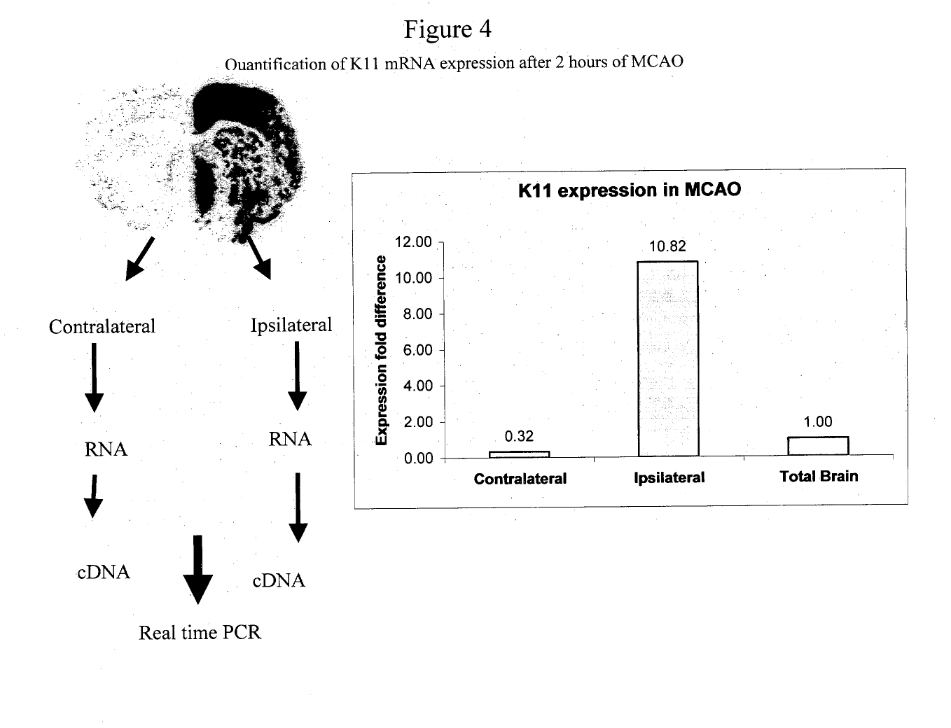

The mRNA expression of K11 was quantified by real time PCR in animals subjected to 2 hours of MCAO and different time of recovery. At 2 hours recovery, a 10 fold over expression of K11 mRNA was detected by real-time PCR analysis in the ischemic hemisphere as compared to the contra lateral hemisphere and the control brain (see FIG. 4).

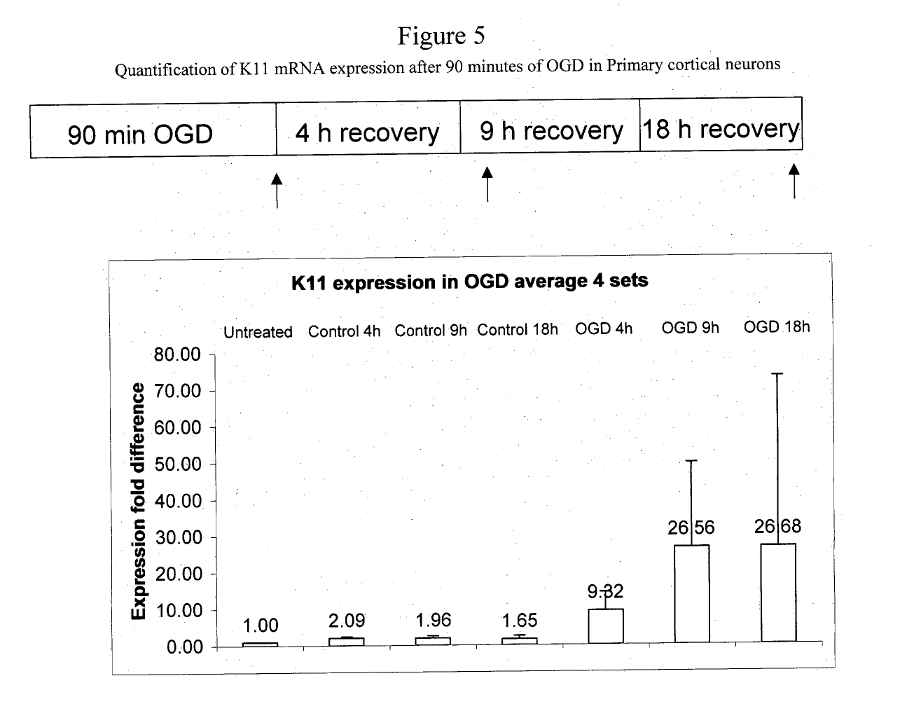

Primary cortical neurons were subjected to 90 min of OGD or sham insult and 4, 9 and 18 hours of recovery (n=3). OGD induced a 10-26-fold induction of this EST in the time points studied (see FIG. 5).

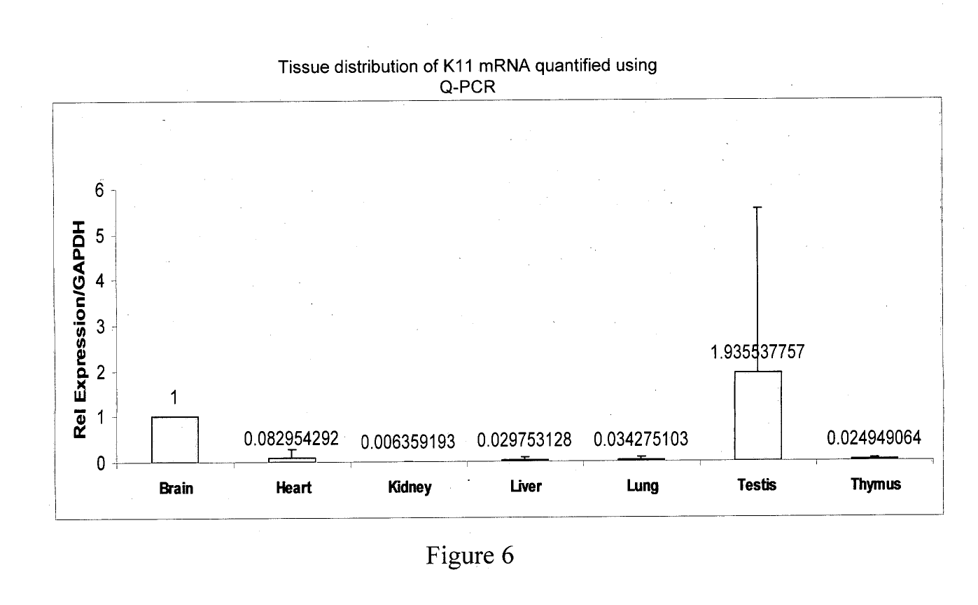

Heart, brain, testis, liver, kidney, thymus, lungs and spleen that were obtained from 6 healthy un-stimulated animals were analyzed for K11 mRNA expression by real-time PCR analysis. K11 mRNA expression is highest in brain and testis, see FIG. 6.

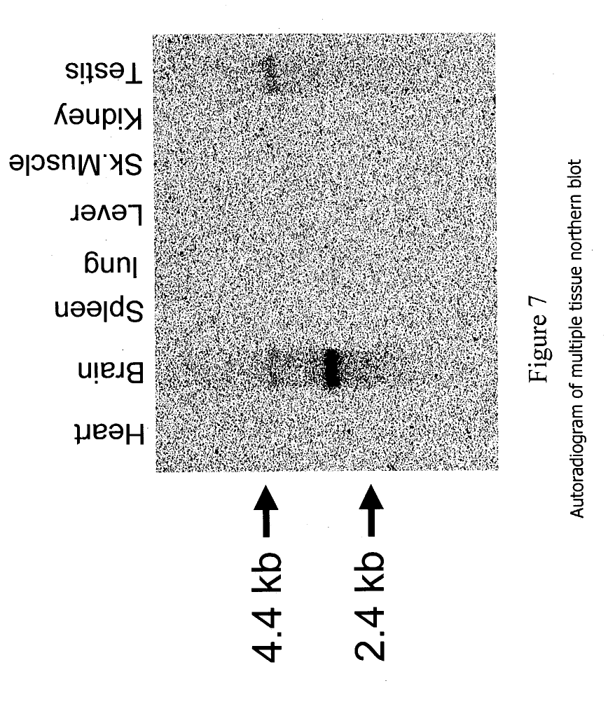

Multiple tissue northern blots were used to profile the expression of K11 in different tissues. In line with our Q-PCR data we find that K11 mRNA was detected in both testis and brain. In the brain a predominant band was detected at about 3.3 kb. However, this band was missing in testis the only band detected in testis was the band at 4.4 kb. (see FIG. 7)

### EXAMPLE 2

Full Length Cloning

5′ and 3′ RACE were used to clone the full-length K11, using Marathon cDNA amplification kit (Clontech) and GeneRacer kit (Invitrogen) according to the manufacturer's instructions. Marathon™ cDNA amplification is a method from Clontech were used for performing both 5′ and 3′ rapid amplification of cDNA ends (RACE) from the same template (Clontech catalog # PT1115-1 (PR15735). Based on the K11 EST sequence, 4 gene specific primers with following seq. were designed and used to do 5′ and 3′ RACE.

The Primers designed for RACE were as follows:

| | |
|---|---|
| Upper primer: | |
| ACTCCACCTTACACACCCCACCAG | (SEQ ID NO:5) |
| ATGAACCCTTCCAGACTCACTTGC | (SEQ ID NO:6) |
| Lower primer: | |
| CTCCTGGGCGAAGTAAGTCTTGGT | (SEQ ID NO:7) |
| AGGGCTCAGTTGCTCTGGGAAGGT | (SEQ ID NO:8) |

Using the lower primer and the clontech Marathon protocol, a 3′ RACE product was obtained which was about 1.8 kb. The 5′ end of the K11 was cloned using the GeneRacer kit from invitrogen (catalog #L1500-01). cDNA were generated according to the geneRacer protocol, using total RNA from ischemic hemisphere of MCAO brain from rat. The cDNA were generated according to the GeneRacer protocol and used in Race reaction. 5′ RACE were conducted and a product of about 2 kb were detected and sequenced. Contig of the both 3′ and 5′ Race resulted in sequence of about 3.3 kb (SEQ ID NO: 1) which is provided in the sequence listing.

Based on the sequencing information, primers were designed to clone the ORF of K11 from both rat and human brain. Genomic seq. of human chromosome 11 was used as template to be able to design cloning primer for human K11.

The rat primers were used in RT_PCR with cDNA template generated from ischemic hemisphere of the brain after 2 hour MCAO. The PCR product was about 2.5 kb long which were cloned in PCR4.0 Topo and sequenced (SEQ ID NO: 5)

Rat primer:

| | | |
|---|---|---|
| Upper: | AtGTACCGATCCACCAAGGGC | (SEQ ID NO:9) |
| Lower: | TCAAAACGTTGGTTCCCCTCC | (SEQ ID NO:10) |

The human primers were used in a RT-PCR with cDNA template generated from human poly A. A 2.5 kb PCR fragment were cloned using this condition (SEQ ID NO: 3).

Human primers:

| | | |
|---|---|---|
| Upper: | ATGTACCGCTCCACCAAGGGCGCC | (SEQ ID NO:11) |
| Lower | TCAAAACGTTGGTTCCCCTCCACTT. | (SEQ ID NO:12) |

### EXAMPLE 3

K11 Specific Antibodies

Materials and Methods

Antisera Preparation. The two antibodies were raised in Zymed custom antibody generation facility. Briefly anti-K11 rabbit polyclonal antisera were generated using either the amino acids 317-334 or c-terminus amino acids 788-802 region of SEQ ID NO: 2. Both antisera were purified with Protein A-Sepharose.

Immunocytochemistry on neurons. Immunohistochemistry on 12 day old neurons was performed. The cells were first fixed in 4% paraformaldehyde for 25 minutes at room temperature and washed with Dulbecco's PBS (dPBS). Cell membranes were ruptured via treatment with 0.5% triton X in 2%BSA/dPBS for 30 minutes at room temperature and blocked with 2-5%BSA in dPBS for 1 hour at room temperature. The cells were incubated with the primary antibody (K11 317 diluted 1:5 in 3% BSA/dPBS or synaptophysin diluted 1/500 in 3% BSA/dPBS) overnight at 4° C. After washing the cells with dPBS the cells were incubated with secondary antibody diluted 1:200 in 3% BSA/dPBS) for one hour at room temperature. Finally the cells were washed with dPBS and visualized under the microscope.

Immunohistochemistry. Perfusion-fixed brains were sectioned on dry ice into 30 μm thick sections with a sliding microtome to obtain free floating sections which were stored in anti-freeze medium (30% etylenglycol, 30% glycerol in 0.5 mol/L phosphate buffer) at −20° C. for later immunostaining procedures. Before staining the sections were treated with 1% hydrogen peroxide for 30 minutes at room temperature to avoid endogenous peroxidase activity. The sections were then washed for 5 minutes in 0.1 mol/L Tris pH 7.6, and in Tris A (0.1 mol/L Tris with 0.1% Triton X) and for 15 minutes in Tris B (0.1 mol/L Tris with 0.1% Triton X and 0.005% BSA). Before overnight incubation at 4° C. with primary anti K11 antibody, the same primary antibody as the Western blot analysis, the sections were incubated with 20% normal serum for one hour at room temperature to block non-specific background. After incubation with primary antibody sections were washed in Tris A and B, 15 minutes in each, and incubated with the secondary antibody for 45 minutes at room temperature. After 15 minutes washes in Tris A and Tris B, sections were incubated in avidin-biotin-horseradish peroxidase complex for one hour at room temperature (Vectastain elite ABC kit). Finally the sections were washed in Tris buffer and stained with 3,3′diaminobenzidine (DAB) for 8-15 minutes (DAB solution: 10 ml 0.1 mol[L Tris, 10 mg DAB, 30 μg gluoxidase, 4 mg ammonium chloride and 20 mg glucose), and washed for about 5 minutes in Tris buffer, prior to dehydration and mounting.

Whole-tissue homogenization. Frozen tissue was homogenized by sonication on ice with an ultrasonic homogenizer (Cole-Palmer Instruments, Chicago, Ill., U.S.A.) at an output setting of 30 for 2×10 sec in 1:10 (mg/μL) homogenization buffer (HB) consisting of 50 mmol/L 3-[N-Morpholino]propane-sulfonic acid (MOPS; pH 7.6), 2 mmol/L dithiotreitol (DTT), 0.1 mmol/L sodium orthovanadate; 3 mmol/L EGTA, 0.1 mmol/L PMSF, 20 μg/mL leupeptin, 5 μg/mL aprotinin, 10 μg/mL pepstatin, 0.5 mmol/L Mg(CH₃COO)₂ and 0.32 mol/L sucrose on ice. The resulting homogenate was suspended in 1:10 (mg/μL) HBT (HB containing 0.2% Triton X-1 00) for 2×10 sec by sonication, and the protein concentration was determined using Bio-Rad DC protein Assay kit (Bio-Rad, Hercules, Calif., U.S.A.) before freezing the samples at −80° C. for later analysis. The protein from the primary cortical neurons were collected in similar manner by using same buffer and discarding the cell debris.

Efectrophoresis and immunoblotting. Electrophoresis was carried out on 8% or 4-20% Novex (Novex Electrophoresis GmbH, Frankfurt, Germany) sodium dodecyl sulphate (SDS) -polyacrylamide gels. The samples (50 μg of total protein) were mixed with a 5×SDS sample buffer consisting of 0.3 mol/L Tris-HCl (pH 6.8), 25% β-mercaptoethanol, 12% SDS, 25 mmol/L EDTA, 20% glycerol, and 0.1% bromphenol blue, boiled for 3 minutes, and subjected to sodium dodecyl sulphate-polyacrylamide gel electrophoresis (SDS-PAGE). Following transfer at a constant voltage of 14 V overnight, the Immobilon-P or nitrocellulose membranes were washed three times with Tris-buffered saline plus 0.1% Tween 20 (TBST; pH 7.4), and then preincubated with a blocking solution of 3% bovine serum albumin in TBST for one hour at room temperature. The membranes were incubated at 4° C. overnight with a primary antibody solution. Following incubation with the primary antibody, the membranes were washed three times with TBST at room temperature and then incubated with an appropriate secondary antibody conjugated with horseradish peroxidase for one hour at 4° C. Finally, the immunoreactive species were visualized using ECL Plus, or Super Signal Western blotting system. (208] The membranes were exposed to a Kodak Bio-max film for various times in order to obtain a range of optical densities linear in relation to the concentration of the proteins to be analyzed. Intermediate exposure times were selected for quantification.

Results

In order to study the K11 two antibody against the following amino acid sequences were designed and generated: K11 317-334; SDTEAWSLRQQLNSENTQ (SEQ ID NO: 13) and K11 788-802: QDSFHEDGSGGEPTF (SEQ ID NO: 14). The two antibodies were raised in Zymed custom antibody generation facility and purified before use.

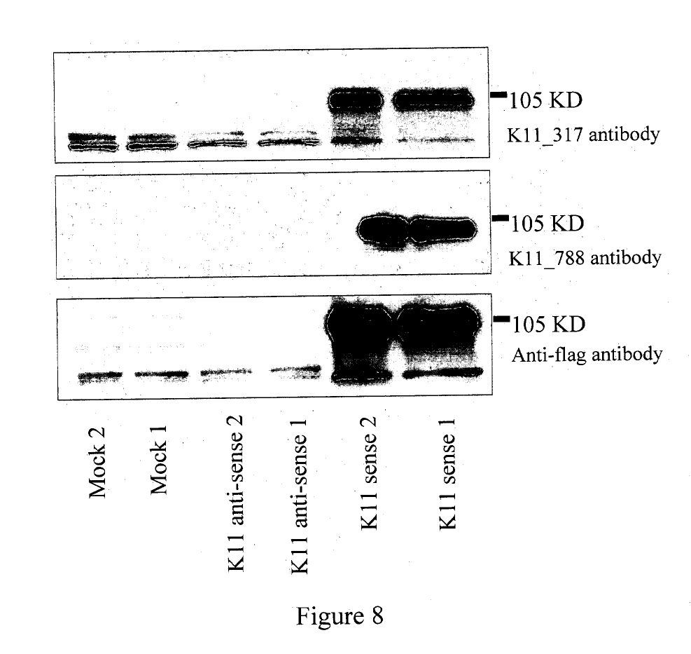

The specificity of the antibodies was verified by overexpressing the FLAG tagged recombinant K11 in HEK 293 cell, as shown in FIG. 8. Tthe FLAG antibody as well as both K11-317 and K11-788 detected a band about 100 kDa. The binding was highly specific, as evidenced by the lack of detectable bands in the antisense and mock transfected cells.

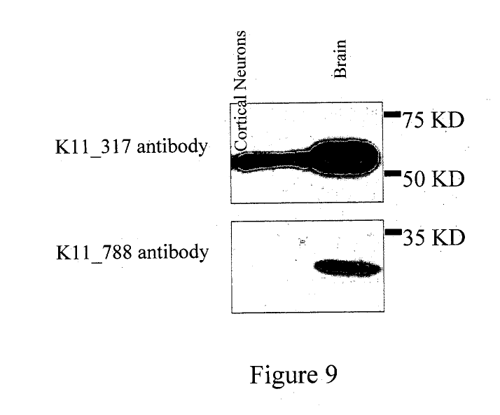

Whole tissue homogenates were prepared from whole rat brain and 14 days old rat primary cortical neurons and were run on 4-20% SDS PAGE and probed with K11_{—}317 and K11_{—}788 antibodies (see FIG. 9). K11_{—}317 detected a band approximately at 60 kDa in both primary cortical neurons and rat brain. When K11_{—}788 antibody was used under the same conditions, a band at approximately 30 kDa was detected, only in brain.

These data indicate that the K11 protein full-length molecular size is about 100 kDa, as was detected when the recombinant protein were overexpressed in 293 cells, see FIG. 8. Moreover, the data shows that the protein is cleaved in the brain, resulting in two fragments at approximately 60 and 30 kDa sizes.

Immunohistochemistry Results

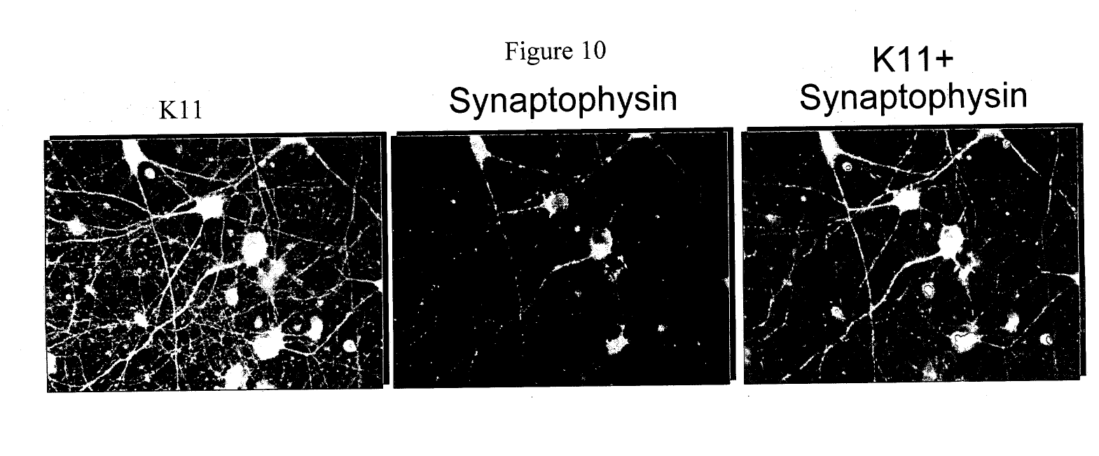

When 12 day old neurons were double stained with both synaptophysin and K11_{—}317 antibodies the following pattern were observed. K11 protein was observed in whole cells. A colocalization with the synaptophysin was observed in synapses however the K11 staining was not exclusively detected in synapses. Data are shown in FIG. 10.

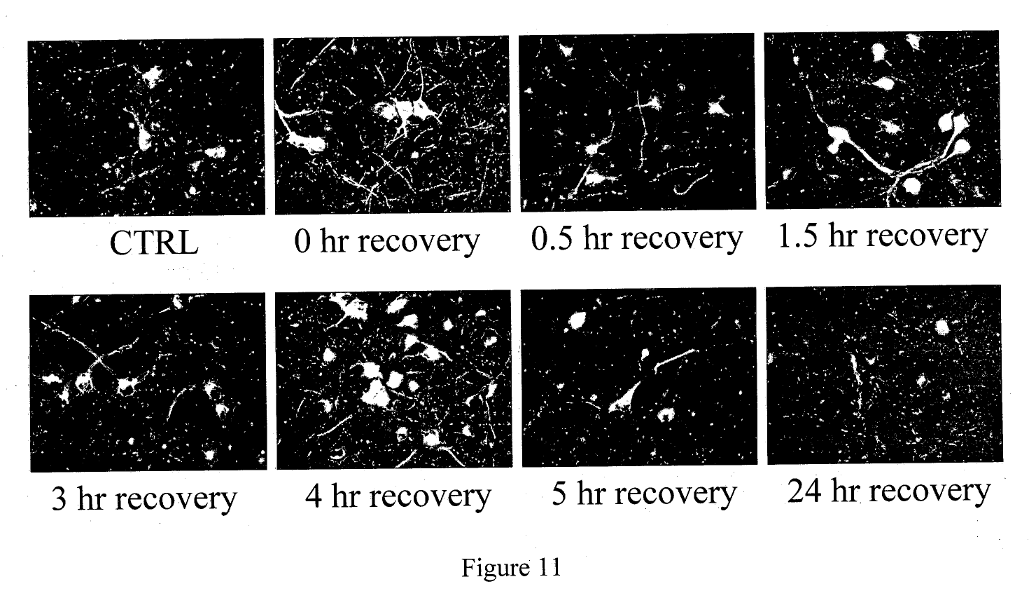

When 12 day old neurons were subjected to 90 min of OGD and 0, 0.5, 1.5, 3, 4, 5 and 24 hours of recovery and stained with K11_{—}317 an upregulation was observed in the neurons at 1.5 and 4 hours after OGD. These data show that K11 plays an essential role in the mechanism of neuronal damage in this model of in vitro ischemia. Data are shown in FIG. 11

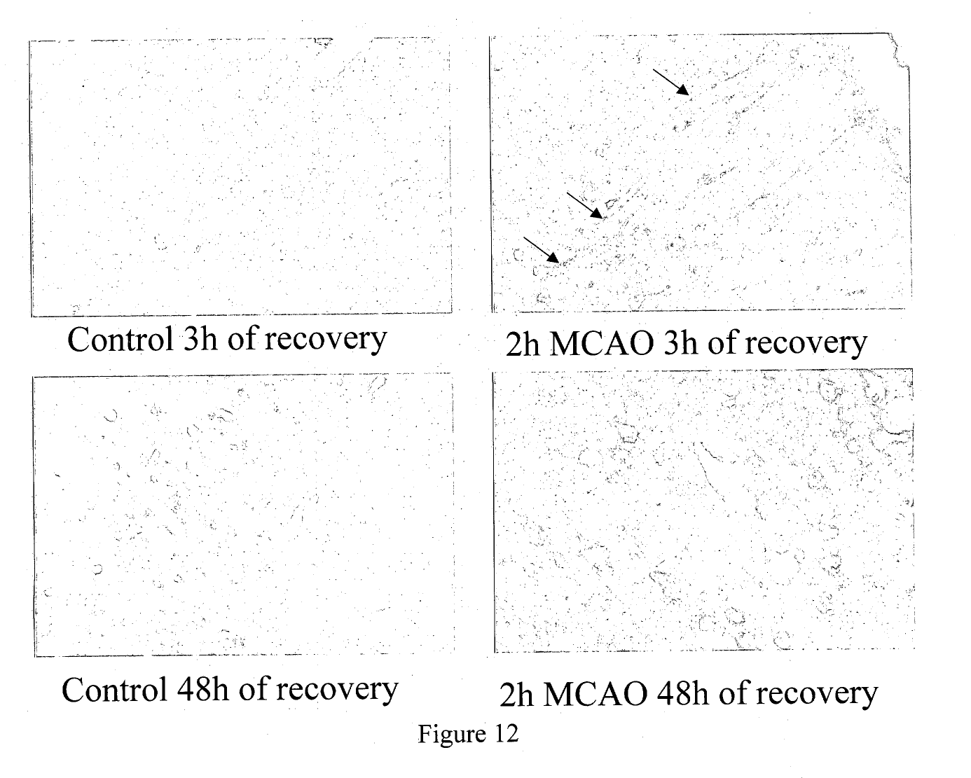

When animals were subjected to 2 hours of MCAO and different times of recovery an upregulation of K11 protein was detected by K11_{—}317 antibody in penumbra as well as ischemic core. The upregulation was observed in the cell body as well as dendrite of cortical neurons. This upregulation of K11 protein was detected in regions of brain that are sensitive to ischemia. (See FIG. 12) This indicates the involvement of this protein in cellular mechanism of ischemic neuronal injury. In physiological condition in control brains, a dense staining was detected in dendrites of the CA1 region of the hippocampus. This indicates the importance of this gene for synaptic transmission and memory formation.

These data show that the mRNA and protein of K11 is altered by both focal and global ischemic insults prior to cell death in the affected regions of the brain. In focal ischemic model an up regulation is present during entire time of reperfusion, which correlates to the protein expression in the affected regions. In global model of ischemic insult when a selective and delayed neuronal damage is present, we detected a persistent up regulation prior to cell death, which indicates that K11 is induced a role in the pathological signaling after ischemic stroke.

The data demonstrate the localization of K11 at dendrites of CA1 region of hippocampus which indicates involvement of this protein in synaptic transmission and memory formation. Therefore, targeting or modulating K11 and proteins regulated by K11 are of great interest for enhancement of memory function in the brain.

Moreover, our data for the first time shows that the K11 is cleaved in adult brain. Based on homology searches, this cleavage is necessary for K11 to be functional. Therefore the responsible protease and nature of cleavage are of great value for the development of therapeutic intervention against stroke.

Finally as described eariler in this application, K11 and other PAS domain proteins are acting as oxygen sensors in the cells. Here we have clearly shown that a novel PAS domain protein is greatly induced in response to ischemia/hypoxia and reperfusion. This indicates that the sensing of oxygen concentration is mediating a critical pathological signaling cascade in ischemic stroke which has not been described before. Development of a method to modulate the activity of a protein involved in this oxygen sensing machinery including K11 but not limited to K11 could be beneficial to therapeutic intervention against stroke, memory dysfunction, other neurodegenerative diseases and finally tools to increase the tolerance against hypoxia in man.

What is claimed is:

1. An isolated nucleic acid molecule encoding the K11 polypeptide set forth in SEQ ID NO: 2 or 4.

2. The isolated nucleic acid according to claim 1, comprising the sequence set forth in SEQ ID NO: 1, 3 or 5.

3. An expression cassette comprising a transcriptional initiation region functional in an expression host, a nucleic acid having a sequence of the isolated nucleic acid according to claim 1 under the transcriptional regulation of said transcriptional initiation region, and a transcriptional termination region functional in said expression host.

4. A cell comprising an expression cassette according to claim 3 as part of an extrachromosomal element or integrated into the genome of a host cell as a result of introduction of said expression cassette into said host cell.

5. An isolated polypeptide comprising the sequence set forth in SEQ ID NO: 2 or 4.

6. The isolated polypeptide of claim 5, wherein said peptide consists of a naturally occurring cleavage product of SEQ ID NO: 2 or 4

7. A method for developing biologically active agents that modulate activity of K11, the method comprising:
combining a candidate biologically active agent with any one of:
(a) the polypeptide as set forth in SEQ ID NO: 2; 4; or 13
(b) a cell comprising a nucleic acid encoding and expressing a polypeptide as set forth in SEQ ID NO: 2; 4; or 13; or
(c) a non-human transgenic animal model for K11 function comprising one of: (i) a knockout of a gene corresponding SEQ ID NO: 1, 3 or 13; (ii) an exogenous and stably transmitted mammalian gene sequence comprising SEQ ID NO: 1, 3 or 13; and
determining the effect of said agent on ischemia induced molecular and cellular changes.

8. The method according to claim 7, wherein said determining step comprises detection of K11 cleavage.

9. The method according to claim 7, wherein said biologically active agent upregulates expression.

10. The method according to claim 7, wherein said biologically active agent downregulates expression.

11. The method according to claim 7, wherein said biologically active agent binds to said polypeptide.

12. The method according to claim 11, wherein said biologically active agent inhibits activity of said polypeptide.

13. The method according to claim 11, wherein said biologically active agent potentiates activity of said polypeptide.

14. A biologically active agent identified by the method according to claim 11.

15. A method for the diagnosis of ischemia, the method comprising:
determining the upregulation of expression of SEQ ID NO: 1, 3 or 13.

16. The method according to claim 15, wherein said ischemia is focal ischemia.

17. The method according to claim 15, wherein said determining comprises detecting the presence of increased amounts of mRNA or polypeptide in neural cells.

18. The method according to claim 15, wherein said determining comprises detecting the presence of increased amounts of polypeptide in blood.

19. An antibody specific for the protein set forth in SEQ ID NO: 2 or SEQ ID NO: 4.


SEQUENCE LISTING (13 sequences)

SEQ ID NO: 1 - DNA, 3333 bp, R. norvegicus
  CDS (184)...(2590)
ggacactgacatggactgaaggagtagaaaagcagcgctgccgagcggagcccaggagtggagcgagagcgagcaagagcctgggcgaaaagaccgggaagcaaggaagaggaagcctccggtgcatcgggaaaggaccgcaggtgttcgggagccggagctggagctccacggccggcagtcatgtaccgatccaccaagggcgcctccaaggcgcgccgcgaccagatcaacgccgagattcggaacctcaaggaactgctgccgttggttgaagcggacaaggtccggctgtcctacctgcacatcatgagtcttgcctgcatctacactcgcaagggtgtcttctttgctggaggcactcctttggctggccccacggggcttctctctgctcaagagcttgaagacatagtggcagcactacctggatttctacttgtgttcacagctgaggggaagttgctatacctgtcggagagtgtgagcgagcatctgggccattctatggtggatctggttgcccagggtgacagtatttacgacatcattgaccctgctgaccatctcactgtgcgccagcagctcaccatgccctctgctctggatgctgatcgccttttccgttgtcgatttaacacatccaagtccctccggcgccagagtgcaggcaacaaactggtgcttattcgaggtcgattccatgctcacccacctggggcctactgggcaggaaaccccgtgttcacagctttctgtgccccactggagccaagaccccgtcccggccctggccctggccctggccctggtcctgcctctctcttcctggccatgttccagagccggcatgctaaggacctagccctactggacatttctgaaagtgtcctaatctacctgggctttgagcgcagcgaactgctctgtaaatcatggtatggactgctacaccccgaggacctggcccacgcttcttctcaacactaccgcctgttggctgaaaatggagatattcaggttgaaatggtggtgagacttcaagccaagcatggaggctggacatggatttactgcatgctatactcggatggtccagaaggccctattactgccaataactaccctatcagtgacacggaagcctggagtcttcgccagcagctaaactctgaaaacacccaggcagcctatgtcctaggaaccccagctgtgctaccctcattctctgagaatgtcttctcccaggagcactgctctaatccactctttacaccagccctggggactcctagaagtgccagcttccccagggcccctgaactaggtgtgatctcaacatcagaagagcttgcccaaccctccaaagaactggacttcagttacctgccattccctgcaaggcctgagccttccctccaagcagacttgagcaaggatttggtgtgtactccaccttacacaccccaccagccaggaggctgcgccttcctcttcagcctccatgaacccttccagactcacttgccccctccatccagctctctccaagaacagctgacgccaagcacggtgactttctctgaacagttgacaccaagcagtgcaaccttcccagatccactaaccagttcactacaaggacagttgactgaaagctcagccagaagctttgaagaacaattgactccgtgcacctctaccttccctgaccagctgcttcccagcactgccacgttcccagaacctctgggtagccccacccatgagcagctgactcctcccagcacagcattccaagcacatctgaacagtcctagccaaaccttcccagagcaactgagccctaatcctaccaagacttacttcgcccaggagggatgcagttttctctatgagaagttgcccccaagtcctagcagccctggtaatggggactgtacactcttggccctagctcaactccggggtcccctctctgtggacgtccccctggtgcctgaaggcctgctcacacctgaggcctctccagtcaagcaaagtttcttccactatacagagaaagagcagaatgagatagatcgtctcatccagcagatcagccagttggctcagggcatggacaggcccttctcagctgaggctggcactggggggctggagccacttggagggctggagcccctgaaccccaacctgtccctgtcaggggctggaccccctgtgcttagcctggatcttaaaccctggaaatgccaggagctggacttcttggttgaccctgataatttattcctggaagagacgccagtggaagacatcttcatggatctttctactccagaccccaatggggaatggggttcaggggatcctgaggcagaggtcccaggagggaccctgtcaccttgcaacaacctgtccccagaagatcacagcttcctggaggacttggccacctatgaaaccgcctttgagacaggagtctcaacattcccctatgaagggtttgctgatgagttgcatcaactccagagccaagttcaagacagcttccatgaagatggaagtggaggggaaccaacgttttgaataagtctgtgacttaacgtcgtcaagtatggcatattgtcatcaagacgtggagccgctctccacccccccgggactgttggggggattctgggggtcagagggggatatatctgattctccaggccctgaaggatttaggggggaggtgggagggcaagggaggggagcaactttttaaaatcaagggacttcgagcgatcccartttccatttcaatctgtattcactcgtagtgagcttccttgaatgggatttcaagcggagaatgggggagtctcacttccccaccgcgttgccccatgggcctgggccagttctccgctcctaggggccaagccacccctaggctttggtgggggaaaggcatggcccacctggggctagcctgtgccctgaggggctcttgacacccacgtagaattctctacaaaccagtaacgggatttcaattccgacggactctgccgccctggcggcccttcctgtgacttttgtgccccgcgcctggggtggggggcgcgaagagacgctacattcctttccgatggaggaaggcagatctgccgtcacacgtgtgcttgcacgagtgcgtgtacctggtgcgggactcacccggccgccagaccgcctaggcttgcccaggtggccacctcgtggtgctgcggtgactttgtagccaactttataataaagtccagtttgcctttttggaaaaaaaaaaaaaaaaaaaaaaaaaaa

SEQ ID NO: 2 - protein, 802 aa, R. norvegicus
MYRSTKGASKARRDQINAEIRNLKELLPLVEADKVRLSYLHIMSLACIYTRKGVFFAGGTPLAGPTGLLSAQELEDIVAALPGFLLVFTAEGKLLYLSESVSEHLGHSMVDLVAQGDSIYDIIDPADHLTVRQQLTMPSALDADRLFRCRFNTSKSLRRQSAGNKLVLIRGRFHAHPPGAYWAGNPVFTAFCAPLEPRPRPGPGPGPGPGPASLFLAMFQSRHAKDLALLDISESVLIYLGFERSELLCKSWYGLLHPEDLAHASSQHYRLLAENGDIQVEMVVRLQAKHGGWTWIYCMLYSDGPEGPITANNYPISDTEAWSLRQQLNSENTQAAYVLGTPAVLPSFSENVFSQEHCSNPLFTPALGTPRSASFPRAPELGVISTSEELAQPSKELDFSYLPFPARPEPSLQADLSKDLVCTPPYTPHQPGGCAFLFSLHEPFQTHLPPPSSSLQEQLTPSTVTFSEQLTPSSATFPDPLTSSLQGQLTESSARSFEEQLTPCTSTFPDQLLPSTATFPEPLGSPTHEQLTPPSTAFQAHLNSPSQTFPEQLSPNPTKTYFAQEGCSFLYEKLPPSPSSPGNGDCTLLALAQLRGPLSVDVPLVPEGLLTPEASPVKQSFFHYTEKEQNEIDRLIQQISQLAQGMDRPFSAEAGTGGLEPLGGLEPLNPNLSLSGAGPPVLSLDLKPWKCQELDFLVDPDNLFLEETPVEDIFMDLSTPDPNGEWGSGDPEAEVPGGTLSPCNNLSPEDHSFLEDLATYETAFETGVSTFPYEGFADELHQLQSQVQDSFHEDGSGGEPTF

SEQ ID NO: 3 - DNA, 2409 bp, Homo sapiens
  CDS (1)...(2409)
atgtaccgctccaccaagggcgcctccaaggcgcgccgggaccagatcaacgccgagatccggaacctcaaggagctgctgccgctggccgaagcggacaaggtccggctgtcctacctgcacatcatgagcctcgcctgcatctacactcgcaagggcgtcttcttcgctggtggcactcctctggcgggccccacggggcttctctcagctcaagagcttgaggacatcgtagcggcactacccggctttctgcttgtgttcacagccgaggggaaattgctctacctgtctgagagtgtgagcgagcatctgggccactccatggtggacctggttgcccagggtgacagcatctacgacatcattgacccagctgaccacctcactgtgcgccagcaactcaccctgccctctgccctggacactgatcgcctcttccgctgccgcttcaacacctccaagtccctcaggcgccagagtgcaggcaacaaactcgtgcttattcgaggccgattccatgctcacccacctggagcctactgggcaggaaatcccgtgttcacagctttctgtgcccctctggagccgagaccccgcccaggtcctggccctggccctggccctgcctcgctcttcctggccatgttccagagccgccatgctaaagacctggctctactggacatctccgagagtgtcctaatctacctgggctttgagcgcagtgaactgctttgtaaatcatggtatggactgctgcaccccgaggacctggcccacgcttctgctcaacactaccgcctgttggctgagagtggagatattcaggcagagatggtggtgaggctacaggccaagactggaggctgggcatggatttactgcctgttatactcagaaggtccagagggacccattactgccaataactacccaatcagtgacatggaagcctggagcctccgccagcagttgaactctgaagacacccaggcggcttatgtcctgggcactccgaccatgctgccctcattccctgaaaacattctttcccaggaagagtgctccagcactaacccactcttcaccgcagcactgggggctcccagaagcaccagcttccccagtgctcctgaactgagtgttgtctctgcatcagaagagcttccccgaccctccaaagaactggacttcagttacctgacattcccttctgggcctgagccttctctccaagcagaactaagcaaggatcttgtgtgcactccaccttacacgccccatcagccaggaggctgtgccttcctcttcagcctccatgagcccttccagacccatttgcccaccccatccagcactcttcaagaacagctgactccaagcactgcgaccttctctgatcagttgacgcccagcagtgcaaccttcccagatccactaactagcccactgcaaggccagttgactgaaacctcggtcagaagctatgaagaccagttgactccctgcacctccaccttcccagaccagctgcttcccagcacagccaccttcccagagcctctgggcagccctgcccatgaacagctgactcctcccagcacagcattccaagcacacctggacagccccagccaaaccttcccagagcaactgagccccaaccctaccaagacttactttgcccaggagggatgcagttttctctatgagaagttgcccccaagtcctagcagccctggtaatggggactgcacgctcttggccctagcccagctccggggccccctctctgtggatgtccccctggtgcccgaaggcctgctcacacctgaggcctctccagtcaagcagagtttcttccactactctgaaaaggagcagaatgagatagaccgtctcatccagcagattagccaattggctcagggcatggacagacccttctcagctgaggctggcactggcggactagagccacttggaggactggagcccctggactccaacctgtccctgtcaggggcaggcccccctgtgctcagcctggacctgaaaccctggaaatgccaggagctggacttcctggctgaccctgataacatgttcctggaagagacgcccgtggaagacatcttcatggatctctctaccccagatcccagtgaggaatggggctcaggggatcctgaggcagagggcccaggaggggccccatcgccttgcaacaacctgtccccagaagaccacagcttcctggaggacctggccacatatgaaaccgcctttgagacaggtgtctcagcattcccctatgatgggtttactgatgagttgcatcaactccagagccaagttcaagacagcttccatgaagatggaagtggaggggaaccaacgttttga

SEQ ID NO: 4 - protein, 802 aa, Homo sapiens
MYRSTKGASKARRDQINAEIRNLKELLPLAEADKVRLSYLHIMSLACIYTRKGVFFAGGTPLAGPTGLLSAQELEDIVAALPGFLLVFTAEGKLLYLSESVSEHLGHSMVDLVAQGDSIYDIIDPADHLTVRQQLTLPSALDTDRLFRCRFNTSKSLRRQSAGNKLVLIRGRFHAHPPGAYWAGNPVFTAFCAPLEPRPRPGPGPGPGPASLFLAMFQSRHAKDLALLDISESVLIYLGFERSELLCKSWYGLLHPEDLAHASAQHYRLLAESGDIQAEMVVRLQAKTGGWAWIYCLLYSEGPEGPITANNYPISDMEAWSLRQQLNSEDTQAAYVLGTPTMLPSFPENILSQEECSSTNPLFTAALGAPRSTSFPSAPELSVVSASEELPRPSKELDFSYLTFPSGPEPSLQAELSKDLVCTPPYTPHQPGGCAFLFSLHEPFQTHLPTPSSTLQEQLTPSTATFSDQLTPSSATFPDPLTSPLQGQLTETSVRSYEDQLTPCTSTFPDQLLPSTATFPEPLGSPAHEQLTPPSTAFQAHLDSPSQTFPEQLSPNPTKTYFAQEGCSFLYEKLPPSPSSPGNGDCTLLALAQLRGPLSVDVPLVPEGLLTPEASPVKQSFFHYSEKEQNEIDRLIQQISQLAQGMDRPFSAEAGTGGLEPLGGLEPLDSNLSLSGAGPPVLSLDLKPWKCQELDFLADPDNMFLEETPVEDIFMDLSTPDPSEEWGSGDPEAEGPGGAPSPCNNLSPEDHSFLEDLATYETAFETGVSAFPYDGFTDELHQLQSQVQDSFHEDGSGGEPTF

SEQ ID NO: 5 - DNA, 24 bp, r. norvegicus
actccaccttacacaccccaccag

SEQ ID NO: 6 - DNA, 24 bp, r. norvegicus
atgaacccttccagactcacttgc

SEQ ID NO: 7 - DNA, 24 bp, r. norvegicus
ctcctgggcgaagtaagtcttggt

SEQ ID NO: 8 - DNA, 24 bp, r. norvegicus
agggctcagttgctctgggaaggt

SEQ ID NO: 9 - DNA, 21 bp, r. norvegicus
atgtaccgatccaccaagggc

SEQ ID NO: 10 - DNA, 21 bp, r. norvegicus
tcaaaacgttggttcccctcc

SEQ ID NO: 11 - DNA, 24 bp, r. norvegicus
atgtaccgctccaccaagggcgcc

SEQ ID NO: 12 - DNA, 25 bp, r. norvegicus
tcaaaacgttggttcccctccactt

SEQ ID NO: 13 - DNA, 2460 bp, R. norvegicus
  CDS (35)...(2443)
gggagccggagctggagctccacggccggcagtcatgtaccgatccaccaagggcgcctccaaggcgcgccgcgaccagatcaacgccgagattcggaacctcaaggaactgctgccgttggctgaagcggacaaggtccggctgtcctacctgcacatcatgagtcttgcctgcatctacactcgcaagggtgtcttctttgctggaggcactcctttggctggccccacggggcttctctctgctcaagagcttgaagacatagtggcagcactacctggatttctacttgtgttcacagctgaggggaagttgctatacctgtcggagagtgtgagcgagcatctgggccattctatggtggatctggttgcccagggtgacagtatttacgacatcattgaccctgctgaccatctcactgtgcgccagcagctcaccatgccctctgctctggatgctgatcgccttttccgttgtcgatttaacacatccaagtccctccggcgccagagtgcaggcaacaaactggtgcttattcgaggtcgattccatgctcacccacctggggcctactgggcaggaaaccccgtgttcacagctttctgtgccccactggagccaagaccccgtcccggccctggccctggccctggccctggtcctgcctctctcttcctggccatgttccagagccggcatgctaaggacctagccctactggacatttctgaaagtgtcctaatctacctgggctttgagcgcagcgaactgctctgtaaatcatggtatggactgctacaccccgaggacctggcccacgcttcttctcaacactaccgcctgttggctgaaaatggagatattcaggctgaaatggtggtgagacttcaagccaagcatggaggctggacatggatttactgcatgctatactcggatggtccagaaggccctattactgccaataactaccctatcagtgacacggaagcctggagtcttcgccagcagctaaactctgaaaacacccaggcagcctatgtcctaggaaccccagctgtgctaccctcattctctgagaatgtcttctcccaggagcactgctctaatccactctttacaccagccctggggactcctagaagtgccagcttccccagggcccctgaactaggtgtgatctcaacatcagaagagcttgcccaaccctccaaagaactggacttcagttacctgccattccctgcaaggcctgagccttccctccaagcagacttgagcaaggatttggtgtgtactccaccttacacaccccaccagccaggaggctgcgccttcctcttcagcctccatgaacccttccagactcacttgccccctccatccagctctctccaagaacagctgacgccaagcacggtgactttctctgaacagttgacaccaagcagtgcaaccttcccagatccactaaccagttcactacaaggacagttgactgaaagctcagccagaagctttgaagaacaattgactccgtgcacctctaccttccctgaccagctgcttcccagcactgccacgttcccagaacctctgggtagccccacccatgagcagctgactcctcccagcacagcattccaagcacatctgaacagtcctagccaaaccttcccagagcaactgagccctaatcctaccaagacttacttcgcccaggagggatgcagttttctctatgagaagttgcccccaagtcctagcagccctggtaatggggactgtacactcttggccctagctcaactccggggtcccctctctgtggacgtccccctggtgcctgaaggcctgctcacacctgaggcctctccagtcaagcaaagtttcttccactatacagagaaagagcagaatgagatagatcgtctcatccagcagatcagccagttggctcagggcatggacaggcccttctcagctgaggctggcactggggggctggagccacttggagggctggagcccctgaaccccaacctgtccctgtcaggggctggaccccctgtgcttagcctggatcttaaaccctggaaatgccaggagctggacttcttggttgaccctgataatttattcctggaagagacgccagtggaagacatcttcatggatctttctactccagaccccaatggggaatggggttcaggggatcctgaggcagaggtcccaggagggaccctgtcaccttgcaacaacctgtccccagaagatcacagcttcctggaggacttggccacctatgaaaccgcctttgagacaggtgtctcaacattcccctatgaagggtttgctgatgagttgcatcaactccagagccaagttcaagacagcttccatgaagatggaagtggaggggaaccaacgttttgaataagtctgtgacttaa